(19) United States
(12) Patent Application Publication
(10) Pub. No.: US 2026/0275259 A1
(43) Pub. Date: Sep. 17, 2026

(54) BIOREACTORS AND SYSTEMS FOR CELL PRODUCTION

(71) Applicant: Stamm Vegh Corporation, San Francisco, CA (US)

(72) Inventors: Javier Nicolás AMADO, Buenos Aires (AR); Juan Francisco LLAMAZARES VEGH, Buenos Aires (AR); Juan Martín CABALEIRO, Buenos Aires (AR); Mariano Gabriel COLLODEL, Buenos Aires (AR); Martín LÓPEZ PALERMO, Buenos Aires (AR); Pablo Gonzalo SALAVERRIA IZAGUIRRE, Buenos Aires (AR); Santiago Jose CARAFI, Buenos Aires (AR)

(21) Appl. No.: 19/670,951

(22) Filed: May 7, 2026

Related U.S. Application Data
(63) Continuation of application No. PCT/US2024/055879, filed on Nov. 14, 2024.
(60) Provisional application No. 63/599,113, filed on Nov. 15, 2023.

(51) Int. Cl. C12M 1/04; C12M 1/34; C12M 3/00; C12M 3/04

(52) U.S. Cl. CPC C12M 23/24; C12M 23/44; C12M 27/10; C12M 41/12; C12M 41/26; C12M 41/32; C12M 41/34; C12M 41/40; C12M 41/44; C12M 41/46

(57) ABSTRACT

Provided are apparatuses, systems, and methods for cell production. In an aspect, the present disclosure provides a bioreactor, comprising: an inlet configured to receive a culture medium and a plurality of cells; a first vertical wall and a second vertical wall defining a cell culture chamber; wherein the first vertical wall comprises a cavity configured to hold the culture medium and the plurality of cells; wherein the second vertical wall comprises a gas permeable membrane; and an outlet configured to direct at least a portion of the culture medium and the plurality of cells out of the cell culture chamber; wherein the cavity has a shape of U or intertwined U.

### CROSS REFERENCE

This application is a continuation of International Application No. PCT/US2024/055879, filed Nov. 14, 2024, which claims the benefit of U.S. Provisional Application No. 63/599,113 filed Nov. 15, 2023, each of which is entirely incorporated herein by reference.

### BACKGROUND

Production of biological products including cells, proteins, and small and large chemical molecules has become an increasing focus in the provision of medical, food, industrial and other types of products. Bioreactors provide an environment for large scale production of cells and for producing proteins and other molecules from such cells. Consistency of the product and the ability to scale production as well as the flexibility to tailor manufacturing to different locations and for environmental conditions are important factors for production.

### SUMMARY

In an aspect, the present disclosure provides a bioreactor, comprising: an inlet configured to receive a culture medium and a plurality of cells; a first vertical wall and a second vertical wall defining a cell culture chamber; wherein the first vertical wall comprises a cavity configured to hold the culture medium and the plurality of cells; wherein the second vertical wall comprises a gas permeable membrane; and an outlet configured to direct at least a portion of the culture medium and the plurality of cells out of the cell culture chamber, wherein the cavity has a shape of U or intertwined U.

In some embodiments, the cavity has a volume of about 5 mL to about 60 mL. In some embodiments, the cavity has a volume of about 6 mL to about 15 mL. In some embodiments, the membrane comprises a material selected from the group consisting of polydimethylsiloxane (PDMS), Teflon, Polytetrafluoroethylene (PTFE), silicon, and coated fabric. In some embodiments, the membrane has a thickness of about 10 micrometers to 500 micrometers. In some embodiments, the cavity has a depth of about 1 mm to about 6 mm. In some embodiments, the second vertical wall comprises a support member, the support member having sufficient stiffness for providing support for the gas permeable membrane. In some embodiments, the support member comprises a material selected from the group consisting of polycarbonate, polymethylmethacrylate, and polyvinylchloride. In some embodiments, the support member comprises a plurality of holes. In some embodiments, the bioreactor further comprises a third vertical wall, wherein the second vertical wall and the third vertical wall define a gas chamber. In some embodiments, the third vertical wall comprises a plurality of cavities. In some embodiments, the bioreactor further comprises a gas inlet and a gas outlet connected to the gas chamber. In some embodiments, the bioreactor further comprises a plurality of sensors. In some embodiments, the plurality of sensors is configured to measure a temperature, medium volume, glucose, glutamate, lactate, pH, ions, osmolarity, cell productivity, cell viability, cell density, pressure, dissolved oxygen level, and/or dissolved CO₂ level inside the cell culture chamber.

In an aspect, the present disclosure provides a bioreactor, comprising: an inlet configured to receive a culture medium and a plurality of cells; a first vertical wall and a second vertical wall defining a cell culture chamber; wherein the first vertical wall comprises a cavity configured to hold the culture medium and the plurality of cells; wherein the second vertical wall comprises a gas permeable membrane; and an outlet configured to direct at least a portion of the culture medium and the plurality of cells out of the cell culture chamber, wherein the gas permeable membrane comprises a PTFE membrane.

In some embodiments, the cavity has a volume of about 5 mL to about 60 mL. In some embodiments, the cavity has a volume of about 6 mL to about 15 mL. In some embodiments, the cavity has a shape that is U, intertwined U, disc-like, or oval. In some embodiments, the membrane has a thickness of about 10 micrometers to 500 micrometers. In some embodiments, the cavity has a depth of about 1 mm to about 6 mm. In some embodiments, the second vertical wall comprises a support member, the support member having sufficient stiffness for providing support for the gas permeable membrane. In some embodiments, the support member comprises polycarbonate. In some embodiments, the support member comprises a plurality of holes. In some embodiments, the bioreactor further comprises a third vertical wall, wherein the second vertical wall and the third vertical wall define a gas chamber. In some embodiments, the third vertical wall comprises a plurality of cavities. In some embodiments, the bioreactor further comprises a gas inlet and a gas outlet connected to the gas chamber. In some embodiments, the bioreactor further comprises a plurality of sensors. In some embodiments, the plurality of sensors is configured to measure a temperature, medium volume, glucose, glutamate, lactate, pH, ions, osmolarity, cell productivity, cell viability, cell density, pressure, dissolved oxygen level, and/or dissolved CO₂ level inside the cell culture chamber.

In an aspect, the present disclosure provides a bioreactor, comprising: an inlet configured to receive a culture medium and a plurality of cells; a first vertical wall and a second vertical wall defining a cell culture chamber; wherein the first vertical wall comprises a cavity configured to hold the culture medium and the plurality of cells; wherein the second vertical wall comprises a gas permeable membrane; and an outlet configured to direct at least a portion of the culture medium and the plurality of cells out of the cell culture chamber, wherein the cavity is configured for minimal dead zone.

In some embodiments, the cavity has a volume of about 5 mL to about 60 mL. In some embodiments, the cavity has a volume of about 6 mL to about 15 mL. In some embodiments, the cavity has a shape of U, intertwined U, disc-like, or oval. In some embodiments, the membrane comprises a material selected from the group consisting of polydimethylsiloxane (PDMS), Teflon, Polytetrafluoroethylene (PTFE), silicone, and coated fabric. In some embodiments, the membrane has a thickness of about 10 micrometers to 500 micrometers. In some embodiments, the cavity has a depth of about 1 mm to about 6 mm. In some embodiments, the second vertical wall comprises a support member, the support member having sufficient stiffness for providing support for the gas permeable membrane. In some embodiments, the support member comprises polycarbonate. In some embodiments, the support member comprises a plurality of holes. In some embodiments, the bioreactor further comprises a third vertical wall, wherein the second vertical wall and the third vertical wall define a gas chamber. In some embodiments, the third vertical wall comprises a plurality of cavities. In some embodiments, the bioreactor further comprises a gas inlet and a gas outlet connected to the gas chamber. In some embodiments, the bioreactor further comprises a plurality of sensors. In some embodiments, the plurality of sensors is configured to measure a temperature, medium volume, glucose, glutamate, lactate, pH, ions, osmolarity, cell productivity, cell viability, cell density, pressure, dissolved oxygen level, and/or dissolved CO₂ level inside the cell culture chamber.

In an aspect, the present disclosure provides a cell culture system, comprising: at least one bioreactor disclosed herein; at least one flipper module configured to rotate the at least one bioreactor; and a harvest and feed module configured to supply a culture medium to the at least one bioreactor and/or take a sample from the at least one bioreactor for analysis.

In some embodiments, the system further comprises an additional sensor configured to measure a temperature and/or humidity of the cell culture system. In some embodiments, one flipper module of the at least one flipper module is configured to rotate one bioreactor of the at least one bioreactor along a horizontal axis. In some embodiments, the system further comprises a temperature controller. In some embodiments, the system further comprises a heater. In some embodiments, the harvest and feed module comprises a tubing support, a L shaped enclosure, a plurality of microchannels, and a plurality of valve caps. In some embodiments, the harvest and feed module comprises a plurality of IR sensors configured to sense a change in the interface. In some embodiments, the plurality of sensors is further configured to monitor a sampling volume. In some embodiments, the harvest and feed module is connected to the at least one bioreactor.

In an aspect, the present disclosure provides a method of growing cells, comprising: (a) inoculating a bioreactor of any one of claims 1-43 with a cell medium comprising at least one cell, and (b) incubating the bioreactor for a period of time under conditions sufficient to permitting cell growth; wherein during the incubating, rotating the bioreactor for a pre-determined angle at pre-determined time periods.

In some embodiments, the rotating is along a horizontal axis. In some embodiments, the rotating comprises (i) rotating the bioreactor by 180° at 30°/s and holding the bioreactor for 4 seconds, (ii) rotating back to original position at 30°/s in a same or opposite direction and holding the bioreactor for 4 s, (iii) rotating the bioreactor at 30°/s by 180° in a same or opposite direction and holding the bioreactor for 4 s, (iv) rotating back at 30°/s to original position in a same or opposite direction and holding the bioreactor for 4 s, and (v) repeating (i)-(iv). In some embodiments, the rotating comprises (i) rotating the bioreactor by 180° at 60°/s and holding the bioreactor for 4 seconds, (ii) rotating back to original position at 60°/s in a same or opposite direction and holding the bioreactor for 4 s, (iii) rotating the bioreactor at 60°/s by 180° in a same or opposite direction and holding the bioreactor for 4 s, (iv) rotating back at 60°/s to original position in a same or opposite direction and holding the bioreactor for 4 s, and (v) repeating (i)-(iv). In some embodiments, the rotating comprises (i) rotating the bioreactor by 180° at 15°/s and holding the bioreactor for 4 seconds, (ii) rotating back to original position at 15°/s in a same or opposite direction and holding the bioreactor for 4 s, (iii) rotating the bioreactor at 15°/s by 180° in a same or opposite direction and holding the bioreactor for 4 s, (iv) rotating back at 15°/s to original position in a same or opposite direction and holding the bioreactor for 4 s, and (v) repeating (i)-(iv). In some embodiments, the cell medium is at most 80% volume of the cell culture chamber. In some embodiments, the period of time is from 1 day to 30 days. In some embodiments, the method further comprises taking a sample from the bioreactor and determining a cell density. In some embodiments, the method further comprises ceasing the incubating when the cell density reaches a pre-determined value.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “figure” and “FIG.” herein), of which:

FIG. 1A shows an expanded view of a bioreactor, FIG. 1B shows an exemplary bioreactor 150 that is assembled, FIG. 1C shows an exemplary assembled bioreactor 160, FIG. 1D shows an exemplary assembled bioreactor 165, and FIGS. 1E-1H show images of exemplary assembled bioreactors with different ports (e.g., inlet and/or outlet) configurations, according to some embodiments;

FIG. 2A shows an exemplary diagram of a cell culture system, FIG. 2B shows an exemplary three-point calibration curve using three different cell concentrations, FIG. 2C shows comparative total cell concentration, FIG. 2D shows an exemplary cell culture system, FIG. 2E shows an image of a part of an exemplary cell culture system 260, FIG. 2F shows an exemplary valve control manifold, and FIG. 2G shows an exemplary pressure control manifold, according to some embodiments;

FIG. 3A shows an exemplary configuration of cell culture systems with external CO₂ tank, compressor, and vacuum pump, FIG. 3B shows an image of a part of an exemplary cell culture system, and FIG. 3C shows an image of a chamber of an exemplary cell culture system, according to some embodiments;

FIG. 4A shows an example flipper module, and FIG. 4B shows example components of a flipper module, according to some embodiments;

FIGS. 5A-5D show components of an example harvest and feed module, and FIG. 5E shows an example harvest and feed module, according to some embodiments;

FIG. 6A shows an example screen of the user interface, FIG. 6B shows an example screen of the H&F analysis tool, and FIGS. 6C and 6D show exemplary temperature and CO₂ data during the operation of the bioreactor, according to some embodiments;

FIG. 7 shows a graph of antibody (Ab) production produced by culturing recombinant cells in the bioreactor, according to some embodiments;

FIG. 8 shows the secretion metabolite dynamics of cells grown in the bioreactor, according to some embodiments;

FIG. 9 shows a computer system 901, according to some embodiments;

FIG. 10A shows an exemplary sampling scheme, FIG. 10B shows average viable cell concentrations (VCC) over time (days) of Chinese Hamster Ovary (CHO) cells for Ceibo systems and control tests, and FIG. 10C shows average cell viability percentage over time (days) of CHO cells for Ceibo systems and control tests, according to some embodiments; and

FIG. 11A shows total cells over time (days) of CHO cells for Ceibo systems and control tests, FIG. 11B shows cell viability over time (days) of CHO cells for Ceibo systems and control tests, FIG. 11C shows total cells and cell viability for control 1 and control 2, and FIG. 11D shows total cells and cell viability for a cell culture system, according to some embodiments.

### DETAILED DESCRIPTION

While various embodiments of the present application have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a,” “an,” and “the” include plural references unless the context clearly dictates otherwise. For example, the term “a sample” includes a plurality of samples, including mixtures thereof.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

“About” with reference to a number refers to that number plus or minus 15% of that number. The term “about” a range refers to that range minus 15% of its lowest value and plus 15% of its greatest value.

The term “bioreactor,” “microbioreactor,” or “cloud microbioreactor (CMB)” as used interchangeably herein, generally refers to a device suitable for growing, culturing and/or storing cells that may include one or more channels or other openings for inputting cells, for providing liquid media and other cell environment factors, and optionally, one or more structures for trapping, containing or directing the flow of cells within the growing/culturing environment of the bioreactor or subsections thereof.

Cell culture systems are usable for growing and produce cells or cell products. Accessibility to culture medium (e.g., nutrients) and oxygen may be critical for the growth of cells. Dead zones in a cell culture system may reduce the efficient space in the cell culture system for cells to grow. In some cases, agitation may be applied to aid in mixing of cells with culture medium and/or diffusion of oxygen in the culture medium. Without sufficient agitation, the cells may sediment and/or aggregate. The sedimentation of cells may reduce the contact of cells with culture medium or oxygen, thereby inhibiting the growth/production of cells or cell products. There is a need for new cell culture systems and bioreactors that can (i) enhance mixing of cells with culture medium, (ii) improve the diffusion of oxygen to the bioreactor, and/or (iii) reduce dead zone of the bioreactor.

Provided herein are systems, components, and methods for producing and maintaining cells and/or for producing and isolating cells and products made by cells, e.g., proteins or antibodies. The systems, components and methods herein provide flexibility to tailor production for different types of cells, types of cellular environments, and types of molecules produced. The systems, components and methods also provide flexibility of scale. For example, the systems, components, and methods described herein may provide for production scale-up without the altering or significantly altering bench-scale growth conditions.

In some embodiments, the system and components comprise one or more bioreactors for growing cells. The bioreactors may be on a microbioreactor scale, such that the system can be constructed as a benchtop bioreactor with a capacity to grow and produce cells and/or cell products in both small and large amounts. This system and method of use are advantageous in their scalability, flexibility, and conservation of resources.

In some embodiments, the systems provided herein may comprise one or more autonomous or semi-autonomous bioreactors used for the optimization of the cell's performance. In some embodiments, the bioreactors may mimic a large scale bioprocessor's setup on a smaller scale. In some embodiments, the bioreactor may be used to analyze a cell's behavior and test different environmental conditions. In some embodiments, the bioreactor may be used to optimize parameters for a specific cell line. The obtained parameters may be applied at a larger scale in the large bioprocessor.

### Bioreactor

In an aspect, the present disclosure provides a bioreactor for the production of cells. The bioreactor may comprise one or more inlets configured to receive a culture medium and/or a plurality of cells. The bioreactor may comprise a first vertical wall and a second vertical wall defining a cell culture chamber. In some embodiments, the first vertical wall may comprise a cavity configured to hold the culture medium and the plurality of cells. In some embodiments, the second vertical wall may comprise a gas permeable membrane. In some embodiments, the gas permeable membrane may not be permeable to liquid (e.g., culture medium). In some embodiments, the bioreactor may provide an environment for the plurality of cells to grow. In some embodiments, cells may be produced from the bioreactor. In some embodiments, the bioreactor may comprise one or more outlets configured to direct at least a portion of the culture medium and/or at least a portion of the produced cells out of the cell culture chamber.

In some embodiments, the cavity may comprise rounded edges (e.g., free of right angles or sharp edges that can accumulate cells or cellular debris). In some embodiments, the cavity may comprise a shape of U, intertwined U, disc-like, annular, or oval. In some embodiments, the cavity may comprise a shape of U. In some embodiments, the two sides of the U shape may be substantially parallel. In some embodiments, the two sides of the U shape may not be parallel. In some embodiments, the two sides of the U shape may have an angle of at least about 1°, at least about 5°, at least about 10°, at least about 15°, at least about 20°, at least about 30°, at least about 45°, or at least about 60°, including increments therein. In some embodiments, the two sides of the U shape may have an angle of at most about 60°, at most about 45°, at most about 30°, at most about 20°, at most about 15°, at most about 10°, at most about 5°, or at most about 1°, including increments therein. In some embodiments, the gas permeable membrane can comprise a material selected from the group consisting of polydimethylsiloxane (PDMS), Teflon, Polytetrafluoroethylene (PTFE) (e.g., as Sterlitech Aspire Laminated QP955), silicone (e.g., SSP M823), and coated fabric. In some embodiments, the gas permeable membrane can comprise a bioink. In some embodiments, the bioink may be 3 dimensional printable. In some embodiments, the bioink may comprise one or more polymerizable components, one or more photoinitiators; and one or more thermocuring agents.

In some embodiments, the one or more polymerizable components may comprise polyethylene glycol diacrylate (PEGDA). In some embodiments, the bioink may comprise about 70% to about 98% (wt/wt) polymerizable components. In some embodiments, the one or more polymerizable components may comprise PEGDA 250 (molecular weight 250). In some embodiments, the one or more photoinitiatiors may comprise 2,2phenylbis(2,4,6-trimethylbenzoyl) phosphine oxide (BAPO). In some embodiments, the bioink may comprise about 2% to about 30% (wt/wt) photoinitiator. In some embodiments, the one or more thermocuring agents may comprise 2,2′-Azobis(2-methylpropionitrile) (AIBN). In some embodiments, the bioink may comprise about 0.01% to about 10% (wt/wt) thermocuring agents.

In some embodiments, the bioink may further comprise a porogen. In some embodiments, the bioink may comprise about 30% to about 70% (wt/wt) porogen. In some embodiments, the porogen may comprise polyethylene glycol. In some embodiments, the porogen may comprise PEG-200 or PEG-400. In some embodiments, the bioink may further comprise a light blocker. In some embodiments, the bioink may comprise about 0.001% to about 1% (wt/wt) light blocker. In some embodiments, the light blocker may comprise Avobenzone (Avo). In some embodiments, the bioink may further comprise a plasticizer. In some embodiments, the bioink may comprise about 15% to about 30% (wt/wt) plasticizer. In some embodiments, the plasticizer may comprise decanol (e.g., n-decanol, decan-1-ol, capric alcohol). In some embodiments, the bioink may further comprise Triton X at a concentration of greater than 10% and less than 50% wt/wt. In some embodiments, the bioink may further comprise a filler. In some embodiments, the bioink may comprise about 1% to about 10% (wt/wt) filler. In some embodiments, the filler may comprise hydroxyapatite. In some embodiments, the hydroxyapatite may comprise modified hydroxyapatite. In some embodiments, the modification of the hydroxyapatite may comprise functionalization with carbon (HApC12)-dodecanol-.

In some embodiments, the bioink may further comprise a rheological enhancer. In some embodiments, the bioink may comprise about 15% to about 30% (wt/wt) rheological enhancer. In some embodiments, the rheological enhancer may comprise decanol (e.g., n-decanol, decan-1-ol, capric alcohol). In some embodiments, the rheological enhancer may comprise SILICA. In some embodiments, the rheological enhancer may comprise modified SILICA. In some embodiments, the modified SILICA may comprise KH550 or (3-aminopropil) trietoxisilano. In some embodiments, the bioink may further comprise a biocompatibility enhancer. In some embodiments, the biocompatibility enhancer may be applied to a surface of the polymerized composition in layers. In some embodiments, the biocompatibility enhancer may comprise between 2 and 12 layers. In some embodiments, the biocompatibility enhancer may comprise at least 6 layers. In some embodiments, the biocompatibility enhancer may comprise polydimethylsiloxane (PDMS). In some embodiments, the polymerized component may comprise at least one surface compatible with growth or maintenance of living cells.

In some embodiments, the cells disclosed herein can be bacterial cells, fungal cells, yeast cells, eukaryotic cells (e.g., mammalian cells, human cells), plant cells, or algal cells. In some embodiments, the cells can be recombinant cells. In some embodiments, the cells can be stem cells.

In some embodiments, the cavity of any of the first vertical wall disclosed herein can have a depth from about 1 millimeter (mm) to about 2 mm, from about 1 mm to about 3 mm, from about 1 mm to about 4 mm, from about 1 mm to about 5 mm, from about 1 mm to about 6 mm, from about 1 mm to about 7 mm, from about 1 mm to about 8 mm, from about 1 mm to about 9 mm, from about 1 mm to about 10 mm, from about 2 mm to about 3 mm, from about 2 mm to about 4 mm, from about 2 mm to about 5 mm, from about 2 mm to about 6 mm, from about 2 mm to about 7 mm, from about 2 mm to about 8 mm, from about 2 mm to about 9 mm, from about 2 mm to about 10 mm, from about 3 mm to about 4 mm, from about 3 mm to about 5 mm, from about 3 mm to about 6 mm, from about 3 mm to about 7 mm, from about 3 mm to about 8 mm, from about 3 mm to about 9 mm, from about 3 mm to about 10 mm, from about 4 mm to about 5 mm, from about 4 mm to about 6 mm, from about 4 mm to about 7 mm, from about 4 mm to about 8 mm, from about 4 mm to about 9 mm, from about 4 mm to about 10 mm, from about 5 mm to about 6 mm, from about 5 mm to about 7 mm, from about 5 mm to about 8 mm, from about 5 mm to about 9 mm, from about 5 mm to about 10 mm, from about 6 mm to about 7 mm, from about 6 mm to about 8 mm, from about 6 mm to about 9 mm, from about 6 mm to about 10 mm, from about 7 mm to about 8 mm, from about 7 mm to about 9 mm, from about 7 mm to about 10 mm, from about 8 mm to about 9 mm, from about 8 mm to about 10 mm, or from about 9 mm to about 10 mm. In some embodiments, the cavity of any of the first vertical wall disclosed herein can have a depth of at least about 1 mm, at least about 2 mm, at least about 3 mm, at least about 5 mm, at least about 10 mm, at least about 50 mm, or at least about 100 mm, including increments therein. In some embodiments, the cavity of any of the first vertical wall disclosed herein can have a depth of at most about 100 mm, at most about 50 mm, at most about 10 mm, at most about 5 mm, at most about 3 mm, at most about 2 mm, or at most about 1 mm, including increments therein.

In some embodiments, the cavity of any of the first vertical wall defines a volume to hold cell culture medium and cells. In some embodiments, the cavity of any of the first vertical wall disclosed herein can have a volume from about 1 milliliter (mL) to about 2 mL, from about 1 mL to about 3 mL, from about 1 mL to about 4 mL, from about 1 mL to about 5 mL, from about 1 mL to about 6 mL, from about 1 mL to about 7 mL, from about 1 mL to about 8 mL, from about 1 mL to about 9 mL, from about 1 mL to about 10 mL, from about 1 mL to about 15 mL, from about 1 mL to about 20 mL, from about 1 mL to about 30 mL, from about 1 mL to about 40 mL, from about 1 mL to about 50 mL, from about 1 mL to about 60 mL, from about 2 mL to about 5 mL, from about 2 mL to about 10 mL, from about 2 mL to about 15 mL, from about 2 mL to about 20 mL, from about 2 mL to about 30 mL, from about 2 mL to about 40 mL, from about 2 mL to about 50 mL, from about 2 mL to about 60 mL, from about 5 mL to about 10 mL, from about 5 mL to about 15 mL, from about 5 mL to about 20 mL, from about 5 mL to about 30 mL, from about 5 mL to about 40 mL, from about 5 mL to about 50 mL, from about 5 mL to about 60 mL, from about 6 mL to about 10 mL, from about 6 mL to about 15 mL, from about 6 mL to about 20 mL, from about 6 mL to about 30 mL, from about 6 mL to about 40 mL, from about 6 mL to about 50 mL, from about 6 mL to about 60 mL, from about 10 mL to about 15 mL, from about 10 mL to about 20 mL, from about 10 mL to about 30 mL, from about 10 mL to about 40 mL, from about 10 mL to about 50 mL, from about 10 mL to about 60 mL, from about 20 mL to about 30 mL, from about 20 mL to about 40 mL, from about 20 mL to about 50 mL, from about 30 mL to about 40 mL, from about 30 mL to about 50 mL, from about 30 mL to about 60 mL, from about 40 mL to about 50 mL, from about 40 mL to about 60 mL, or from about 50 mL to about 60 mL. In some embodiments, the cavity can have a volume of about 1 mL, about 2 mL, about 3 mL, about 4 mL, about 5 mL, about 6 mL, about 7 mL, about 8 mL, about 9 mL, about 10 mL, about 15 mL, about 16 mL, about 20 mL, about 30 mL, about 40 mL, about 50 mL, or about 60 mL, including increments therein. In some embodiments, the volume can be at least about 1 mL, at least about 5 mL, at least about 10 mL, at least about 50 mL, at least about 100 mL, or at least about 1 L, including increments therein. In some embodiments, the volume can be at most about 1 L, at most about 100 mL, at most about 50 mL, at most about 10 mL, or at most about 1 mL, including increments therein.

In some embodiments, the gas permeable membrane of any of the bioreactor disclosed herein may be integrated to the second vertical wall. Alternatively, or in addition to, the gas permeable membrane may be separate from the second vertical wall.

In some embodiments, oxygen can be permeated through the gas permeable membrane for replenishing oxygen in the culture medium. In some embodiments, carbon dioxide can be permeated through the gas permeable membrane to maintain a required level of carbon dioxide in the culture medium.

In some embodiments, the diffusion of the oxygen in a culture medium can take a long time and the partial pressures or the partial pressure gradient of the oxygen in the culture medium decreases as a function of distance from the gas permeation membrane. In some embodiments, the bioreactor may be configured to be rotated, rocked, shaken, or flipped during the growth/production of the cells. In some embodiments, the rotation, rocking, shaking, or flipping of the bioreactor can prevent the sedimentation of the cells. In some embodiments, the rotation, rocking, or flipping of the bioreactor can resuspend the cells. In some embodiments, the rotation, rocking, shaking, or flipping of the bioreactor can provide slight agitation to the culture mixture, enhancing the diffusion and distribution of oxygen in the culture medium.

In some embodiments, the gas permeable membrane of any of the bioreactor disclosed herein may be configured to cover the cavity of the first vertical wall. The gas permeable membrane may not be permeable to the culture media or the cells such that only gas can penetrate the membrane while the culture media or cells do not. In some embodiments, the gas permeable membrane may have any shape or dimension as long as it can cover the cavity of the first vertical wall. In some embodiments, the gas permeable membrane can have a shape that is substantially similar to the shape of the cavity. In some embodiments, the gas permeable membrane can have a size that is at least about 5%, at least about 10%, at least about 15%, at least about 20%, at least about 25%, at least about 30%, or at least about 40% larger than the size of the cavity in the vertical plane. In some embodiments, the gas permeable membrane may have a thickness from about 10 micrometers (μm) to about 20 μm, from about 10 μm to about 30 μm, from about 10 μm to about 40 μm, from about 10 μm to about 50 μm, from about 10 μm to about 60 μm, from about 10 μm to about 70 μm, from about 10 μm to about 80 μm, from about 10 μm to about 90 μm, from about 10 μm to about 100 μm, from about 10 μm to about 200 μm, from about 10 μm to about 300 μm, from about 10 μm to about 400 μm, from about 10 μm to about 500 μm, from about 20 μm to about 30 μm, from about 20 μm to about 40 μm, from about 20 μm to about 50 μm, from about 20 μm to about 60 μm, from about 20 μm to about 70 μm, from about 20 μm to about 80 μm, from about 20 μm to about 90 μm, from about 20 μm to about 100 μm, from about 20 μm to about 200 μm, from about 20 μm to about 300 μm, from about 20 μm to about 400 μm, from about 20 μm to about 500 μm, from about 30 μm to about 30 μm, from about 30 μm to about 50 μm, from about 30 μm to about 60 μm, from about 30 μm to about 70 μm, from about 30 μm to about 80 μm, from about 30 μm to about 90 μm, from about 30 μm to about 100 μm, from about 30 μm to about 200 μm, from about 30 μm to about 300 μm, from about 30 μm to about 400 μm, from about 30 μm to about 500 μm, from about 50 μm to about 100 μm, from about 50 μm to about 200 μm, from about 50 μm to about 300 μm, from about 50 μm to about 400 μm, from about 50 μm to about 500 μm, from about 100 μm to about 200 μm, from about 100 μm to about 300 μm, from about 100 μm to about 400 μm, from about 100 μm to about 500 μm, from about 200 μm to about 300 μm, from about 200 μm to about 400 μm, from about 200 μm to about 500 μm, from about 300 μm to about 400 μm, from about 300 μm to about 500 μm, or from about 400 μm to about 500 μm.

In some embodiments, the second vertical wall of any of the bioreactor disclosed herein may comprise a support member having sufficient stiffness for providing support for the gas permeable membrane. When the bioreactor is filled with liquid, e.g., the culture medium, the bottom portion of the gas permeable membrane can become slightly distorted from the weight of the liquid in the bioreactor and the bottom portion of the gas permeable membrane may be pushed more against the second vertical wall at the bottom and pushed less at the top portion of the second vertical wall due to hydrostatic pressure of the liquid. The support member can provide a counterforce on the gas permeable membrane to maintain the shape of the gas permeable membrane. In some embodiments, the second vertical wall of any of the bioreactor disclosed herein may not comprise a support member.

In some embodiments, the support member may comprise a material selected from the group consisting of polycarbonate, polymethylmethacrylate, and polyvinylchloride. In some embodiments, the support member may comprise a plurality of holes.

In some embodiments, the bioreactor disclosed herein may comprise a third vertical wall, wherein the second vertical wall and the third vertical wall define a gas chamber. In some embodiments, the third vertical wall comprises a plurality of cavities. In some embodiments, the bioreactor may comprise a gas inlet and a gas outlet connected to the gas chamber. In some embodiments, the bioreactor disclosed herein may not comprise a third vertical wall.

In some embodiments, the bioreactor may have at least 1, at least 2, at least 3, at least at least 4, at least 5, at least 6, at least 7, at least 8, at least 9, at least 10, or more input channels. In some embodiments, the bioreactor may have at least 1, at least 2, at least 3, at least at least 4, at least 5, at least 6, at least 7, at least 8, at least 9, at least 10, or more output channels. In some cases, during the cell growth, nutrients in the cell culture chamber can be depleted before the culture growth process is completed and waste products of the cells can accumulate to undesirable concentrations in the cell culture chamber. In some embodiments, the one or more input channels can be configured to feed or replenish fresh culture medium and/or cells to the bioreactor. In some embodiments, the one or more output channels can be configured to direct the waste products out of the bioreactor.

In some embodiments, the bioreactor may further comprise one or more sensors. In some embodiments, the one or more sensors are configured to measure a parameter of the culture, the cells, and/or the environment, such as temperature, medium volume, glucose, glutamate, lactate, pH, ions (e.g., Na⁺, NH₄⁺, Ca²⁺ or K⁺), osmolarity, cell productivity, cell viability, cell density, pressure, dissolved oxygen level, and/or dissolved CO₂ level inside the cell culture chamber.

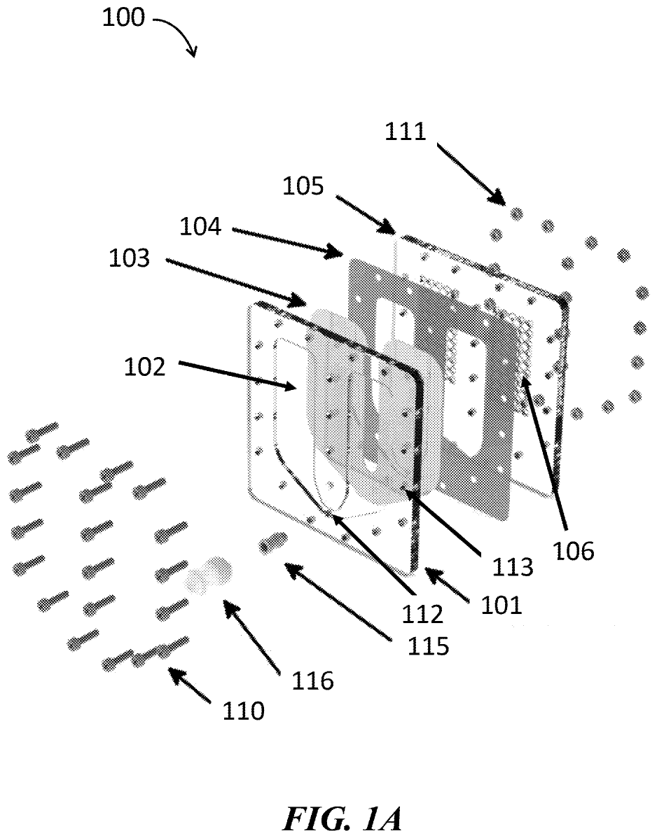

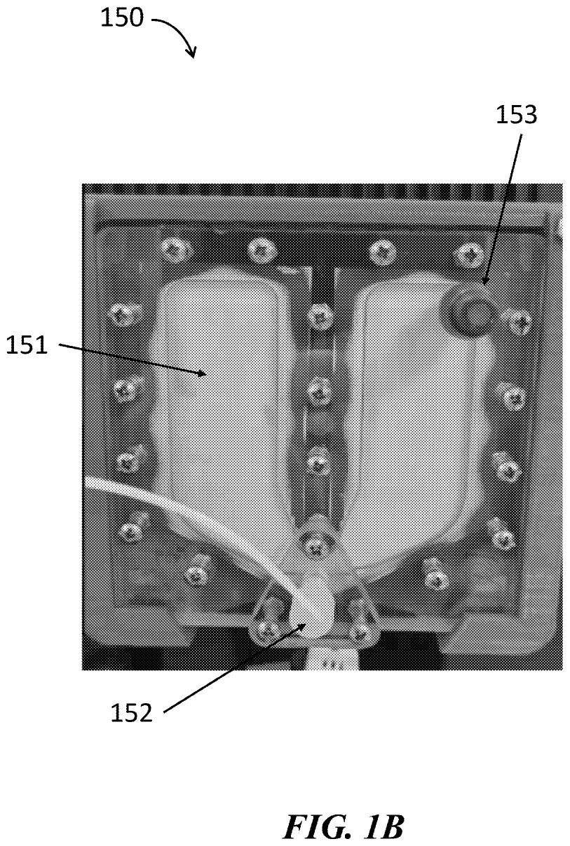

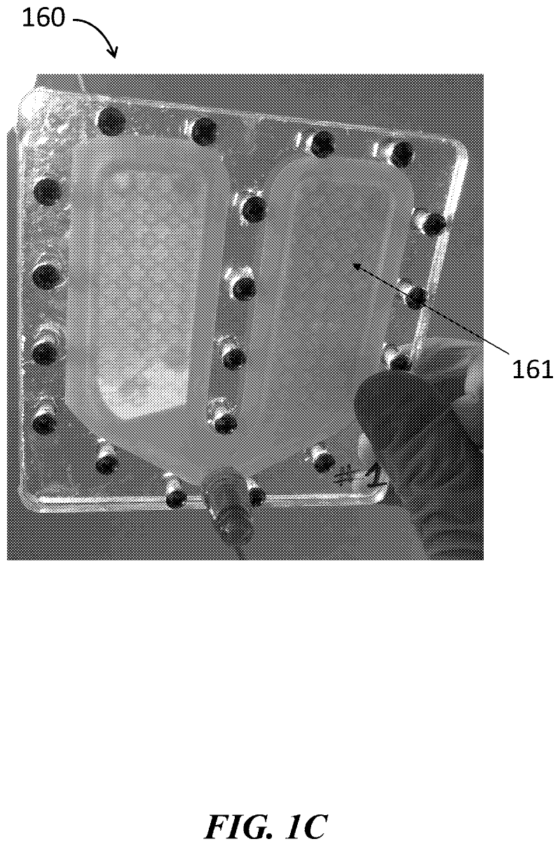

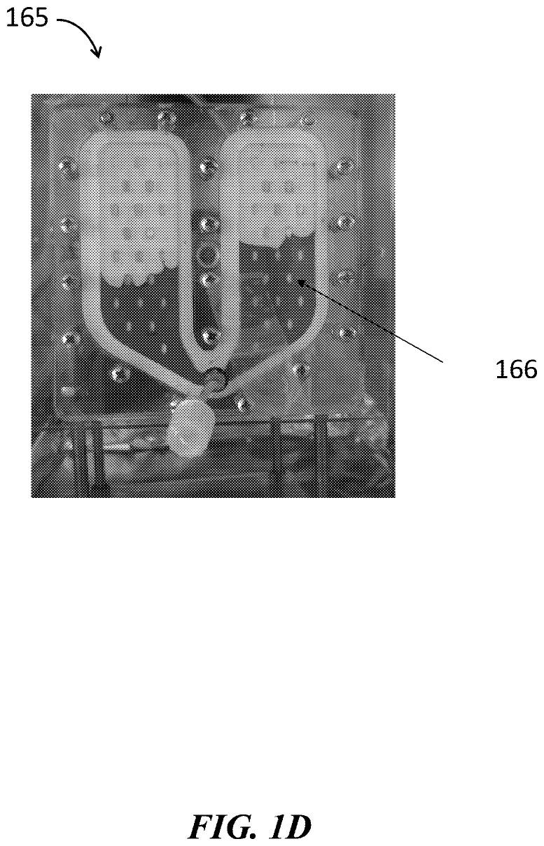

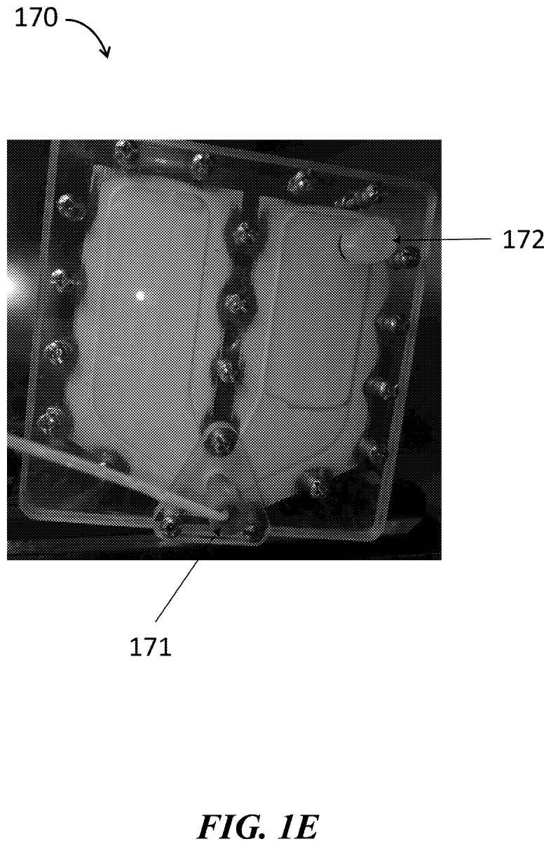

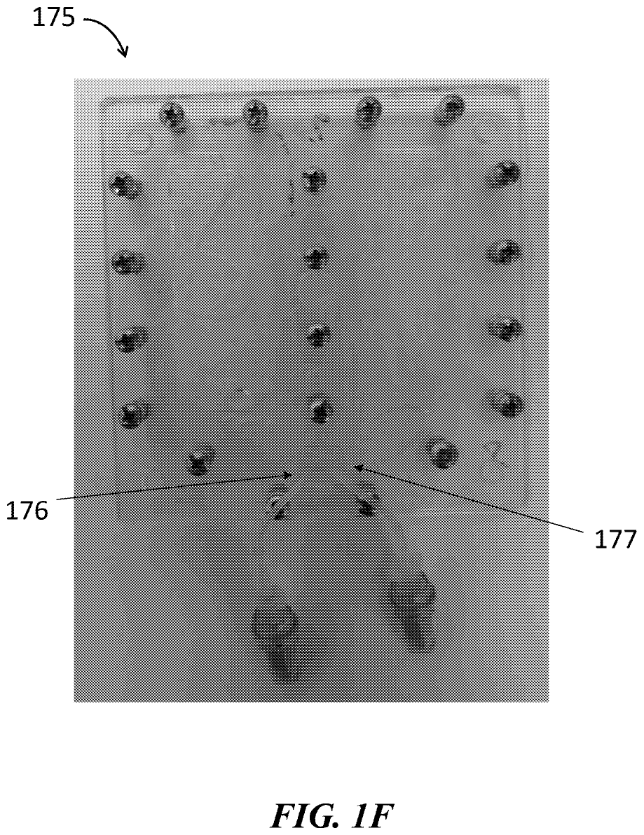

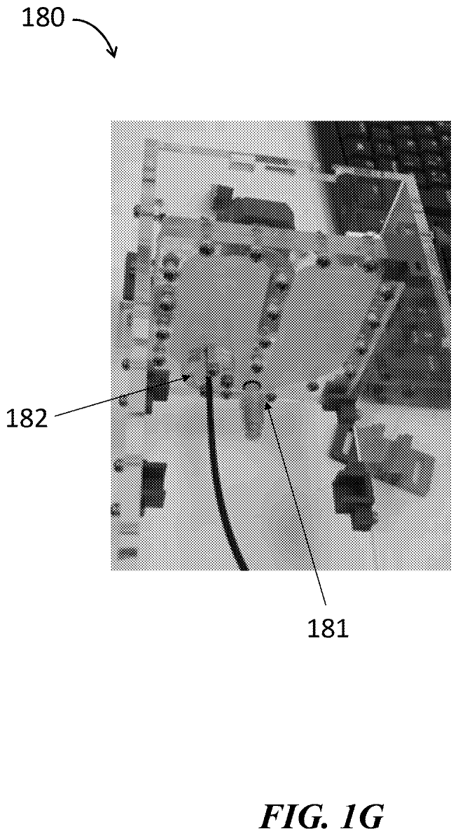

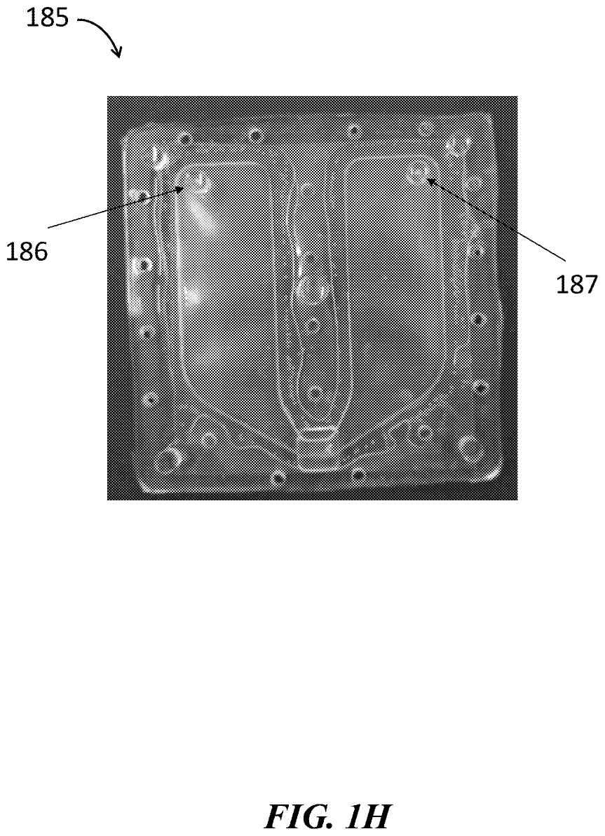

FIG. 1A shows an expanded view of a bioreactor. The bioreactor 100 comprises a first vertical wall 101 comprising a U-shaped cavity 102 for trapping or holding a culture medium and cells, a gas permeable membrane 103, a second vertical wall 104, and a third vertical wall 105 comprising a plurality of cavities 106. The gas permeable membrane 103 is sandwiched in between the first vertical wall 101 and the second vertical wall 104, covering the cavity 102 of the first vertical wall 101. The first vertical wall 102, the membrane 103, the second vertical wall 104, and the third vertical wall 105 can be assembled by a plurality of screws (e.g., 110) and nuts (e.g., 111) through a plurality of holes (e.g., 113) distributed at the non-cavity area. The first vertical wall 101 further comprises a hole at the connecting part of the U-shaped cavity, serving as an inlet 112 for feeding a culture media and cells. The inlet can comprise a connector 115 (e.g., a female Luer connector) and an adaptor 116 for connecting to external tubing. In some embodiments, the inlet may be dually function as an outlet. In some embodiments, the bioreactor 100 may further comprise one or more outlets. In some embodiments, an outlet may be configured to direct culture medium and/or cells out of the bioreactor or for sampling during the operation of the bioreactor.

FIG. 1B shows an exemplary bioreactor 150 that is assembled. The bioreactor 150 comprises a U-shaped cavity 151 in the first vertical wall, an inlet 152 for delivering culture medium and/or cells to the bioreactor, an outlet 153 for directing culture medium and/or cells out of the bioreactor or for sampling during the operation of the bioreactor.

In some embodiments, the first vertical wall may comprise a plurality of posts. In some embodiments, the post may have a height that is substantially similar to the depth of the cavity. In some embodiments, the post may have a height that is at least about 1%, at least about 2%, at least about 5%, at least about 10%, or at least about 20% less than the depth of the cavity, including increments therein. In some embodiments, the post may have a height that is at most about 20%, at most about 10%, at most about 5%, at most about 2%, or at most about 1% less than the depth of the cavity, including increments therein. In some embodiments, the post may have a height that is at least about 1%, at least about 2%, at least about 5%, at least about 10%, or at least about 20% larger than the depth of the cavity, including increments therein. In some embodiments, the post may have a height that is at most about 20%, at most about 10%, at most about 5%, at most about 2%, or at most about 1% larger than the depth of the cavity, including increments therein. In some embodiments, the posts may be configured to maintain the distance between the first vertical wall and the membrane. In some embodiments, the posts may be configured to prevent the membrane from deflecting inward. In some embodiments, the posts may be configured to prevent the membrane from collapsing inward. In some embodiments, the posts may be configured to improve turbulence of the medium when the bioreactor is rocked, rotated, shaken, or flipped. In some embodiments, the posts may be configured to aid in mixing of the medium and cells. In some embodiments, the posts may be configured to increase diffusion of gases in the medium. FIG. 1C shows an exemplary assembled bioreactor 160. The bioreactor may comprise a U shape. The bioreactor 160 may comprise a plurality of posts (e.g., 161). FIG. 1D shows an exemplary assembled bioreactor 165. The bioreactor may comprise a U shape. The bioreactor 165 may comprise a plurality of posts (e.g., 166).

FIGS. 1E-1H show images of exemplary assembled bioreactors with different ports (e.g., inlet and/or outlet) configurations. FIG. 1E shows a port 171 at the connection area of the U shape and an additional port 172 at one arm of the U shape of the reactor 170. FIG. 1F shows two ports 176 and 177 at the connection area of the U shape of the reactor 175. FIG. 1G shows a port 181 at the connection area of the U shape and an additional port 182 at one arm of the U shape of the reactor 180. FIG. 1H shows a port 186 at one arm of the U shape and an additional port 187 at another arm of the U shape of the reactor 185.

In some embodiments, the bioreactor can be operated in a continuous mode, e.g., with culture medium and/or cells feeding in or extracting out of the bioreactor continuously. In some embodiments, the bioreactor can be operated in a batch mode. In some embodiments, the bioreactor can be operated in a semi-batch mode.

In some embodiments, the materials that make up the bioreactor are biocompatible. In some embodiments, the materials that make up the bioreactor are sterilizable. In some embodiments, the bioreactor or the materials making up the bioreactor can be sterilized by autoclave or ethylene oxide.

In an aspect, the present disclosure provides a bioreactor, comprising: (i) an inlet configured to receive a culture medium and a plurality of cells; (ii) a first vertical wall and a second vertical wall defining a cell culture chamber, wherein the first vertical comprising a cavity configured to hold the culture medium and the plurality of cells and the second vertical wall comprises a gas permeable membrane; and (iii) an outlet configured to direct at least a portion of the culture medium and the plurality of cells out of the cell culture chamber, wherein the cavity is configured for minimal dead zone. In some embodiments, the gas permeable membrane can comprise a material selected from the group consisting of polydimethylsiloxane (PDMS), Teflon, Polytetrafluoroethylene (PTFE) (e.g., Sterlitech Aspire Laminated QP955), silicone (e.g., SSP M823), and coated fabric. In some embodiments, the cavity has a shape of U, intertwined U, disc-like, or oval.

In some embodiments, minimal dead zone may be achieved by a design of the cavity and a predefined sequence of rotation of the bioreactor. In some embodiments, the predefined sequence of rotation may have a non-zero velocity all along the cavity and lead to an efficient mixing in every zone of the cavity. In some embodiments, the cavity may have the volume partially occupied by the cell culture and part by air, leading to a constant mix and exchange due to the bubbles of air.

In an aspect, the present disclosure provides a bioreactor for the production of cells. The bioreactor may comprise (i) an inlet configured to receive a culture medium and a plurality of cells; (ii) a first vertical wall and a second vertical wall defining a cell culture chamber, wherein the first vertical wall comprises a cavity configured to hold the culture medium and the plurality of cells and the second vertical wall comprises a gas permeable membrane that is not permeable to liquid (e.g., culture medium); and (iii) an outlet configured to direct at least a portion of the culture medium and the plurality of cells out of the cell culture chamber, wherein the cavity has a shape of U or intertwined U in the vertical plane. In some embodiments, the gas permeable membrane can comprise a material selected from the group consisting of polydimethylsiloxane (PDMS), Teflon, Polytetrafluoroethylene (PTFE) (e.g., as Sterlitech Aspire Laminated QP955), silicone (e.g., SSP M823), and coated fabric.

In an aspect, the present disclosure provides a bioreactor, comprising: (i) an inlet configured to receive a culture medium and a plurality of cells; (ii) a first vertical wall and a second vertical wall defining a cell culture chamber, wherein the first vertical wall comprises a cavity configured to hold the culture medium and the plurality of cells and the second vertical wall comprises a gas permeable membrane; and (iii) an outlet configured to direct at least a portion of the culture medium and the plurality of cells out of the cell culture chamber, wherein the gas permeable membrane comprises a PTFE membrane. In some embodiments, the cavity has rounded edges (e.g., free of right angles or sharp edges that can accumulate cells or cellular debris). In some embodiments, the cavity has a shape of U, intertwined U, disc-like, or oval.

### Cell Culture System

In an aspect, the present disclosure provides a cell culture system, comprising: (i) at least one bioreactor disclosed herein; (ii) at least one flipper module configured to rotate the at least one bioreactor; and (iii) a harvest and feed module configured to supply a culture medium to the at least one bioreactor and/or take a sample from the at least one bioreactor for analysis.

In some embodiments, the bioreactor disclosed in the present disclosure may be placed in a cell culture system. In some embodiments, the cell culture system may comprise a production chamber. In some embodiments, the production chamber may be configured to encase the bioreactor. In some embodiments, the production chamber may be configured to provide humidity control for the bioreactor. In some embodiments, the production chamber may be filled with a gas or a mixture of gases (e.g., a plurality of gases, or one or more gases). In some embodiments, the production chamber may be configured to provide the gas sources to the bioreactor. In some embodiments, the production chamber may have a pressure from 0 to 1 bar. In some embodiments, the production chamber may have a humidity from 0 to 100%. In some embodiments, the production chamber may have a humidity from 50% to 95%.

In some embodiments, the production chamber may comprise a prismatic shape. In some embodiments, the production chamber may comprise a rectangular prism shape. In some embodiments, the production chamber may comprise an opening via clamps on all dorsal sides.

In some embodiments, the production chamber may comprise a harvest and feed module. In some embodiments, the harvest and feed module may be encased inside the production chamber. In some embodiments, the harvest and feed module may be placed outside of the production chamber.

In some embodiments, the production chamber may comprise a cylinder shape. In some embodiments, the production chamber may comprise two sealing planes. In some embodiments, the production chamber may comprise a sealing plane at the front face of the production chamber. In some embodiments, the production chamber may comprise a sealing plane at the rear face of the production chamber. In some embodiments, the production chamber may comprise a double-cylinder sealing system. In some embodiments, the double-cylinder sealing system may be configured to create an air cushion. In some embodiments, the air cushion may thermally insulate the production chamber and/or the bioreactor. In some embodiments, the air cushion may improve condensation inside the production chamber. In some embodiments, the air cushion may enhance thermal efficiency of the system. In some embodiments, the production chamber may comprise a removable rear cover, allowing easy maintenance and removal of components from the production chamber. In some embodiments, the production chamber may comprise a front door with a hinge opening. In some embodiments, the front door may be equipped with a rubber gasket that allows the passage of hoses without compromising the system's seal and sterility.

In some embodiments, one flipper module of the at least one flipper module may be configured to rotate one bioreactor of the at least one bioreactor along a horizontal axis. In some embodiments, the cell culture system may comprise one or more flipper control modules. In some embodiments, the one or more flipper control modules may be configured to control the flipper to go to a position or operate according to a sequence. In some embodiments, the one or more flipper control modules may send commands or sequence instructions to the flipper modules to operate the bioreactor.

In some embodiments, the cell culture system may further comprise an additional sensor configured to measure a temperature and/or humidity of the cell culture system. In some embodiments, the cell culture system may further comprise a temperature controller.

In some embodiments, the cell culture system may further comprise a heater to supply energy to the cell culture system and/or maintain a temperature suitable for cell growth.

In some embodiments, the harvest and feed module may comprise a tubing support, a L shaped enclosure, a plurality of microchannels, and a plurality of valve caps.

In some embodiments, the harvest and feed module may comprise a plurality of infrared (IR) sensors configured to sense a change in the interface. In some embodiments, the harvest and feed module may comprise a sensor to monitor a sampling volume.

In some embodiments, the harvest and feed module may be connected to the at least one bioreactor. In some embodiments, the harvest and feed module may be connected to one or more bioreactors. In some embodiments, the harvest and feed module may be connected to one or more culture media reservoirs. In some embodiments, the harvest and feed module may be connected to a treated water reservoir. In some embodiments, the harvest and feed module may be connected to a solvent (e.g., 70% ethanol, 96% ethanol) reservoir.

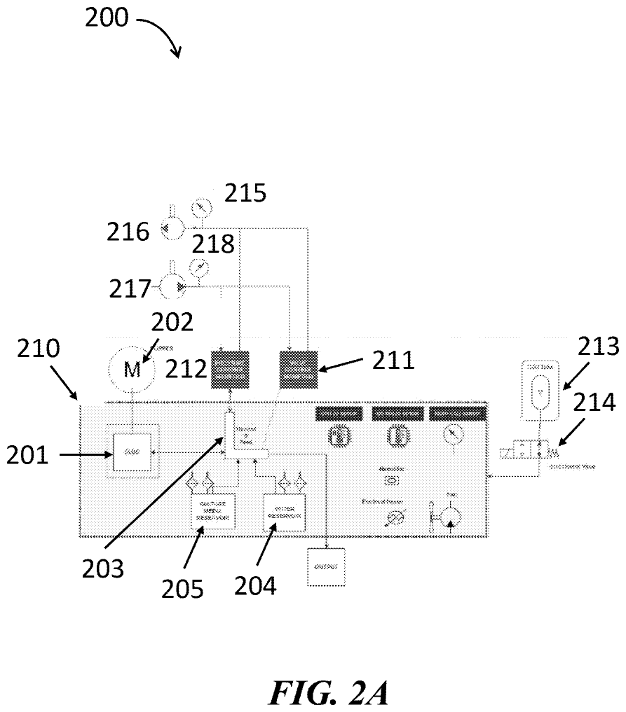

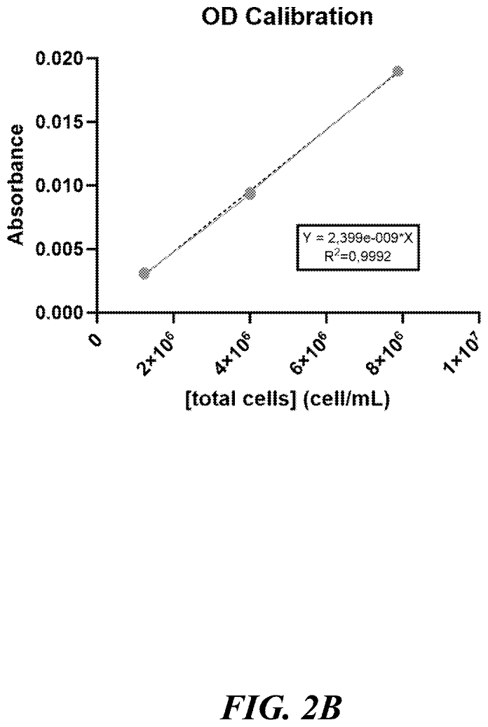

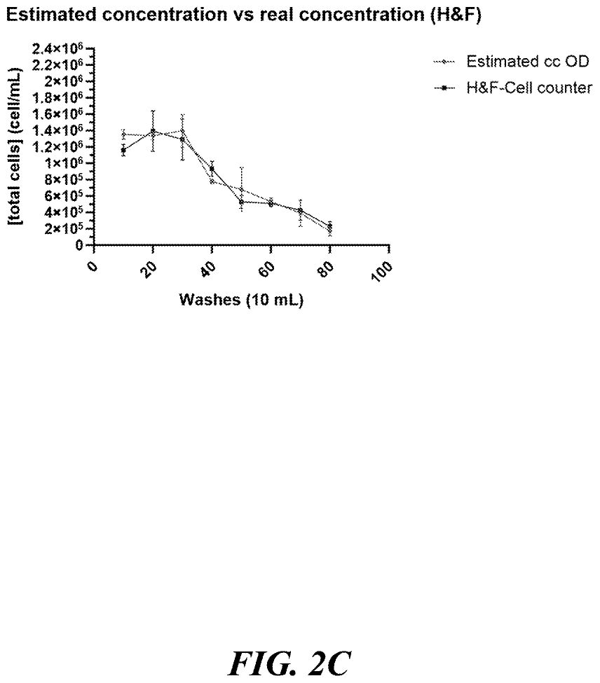

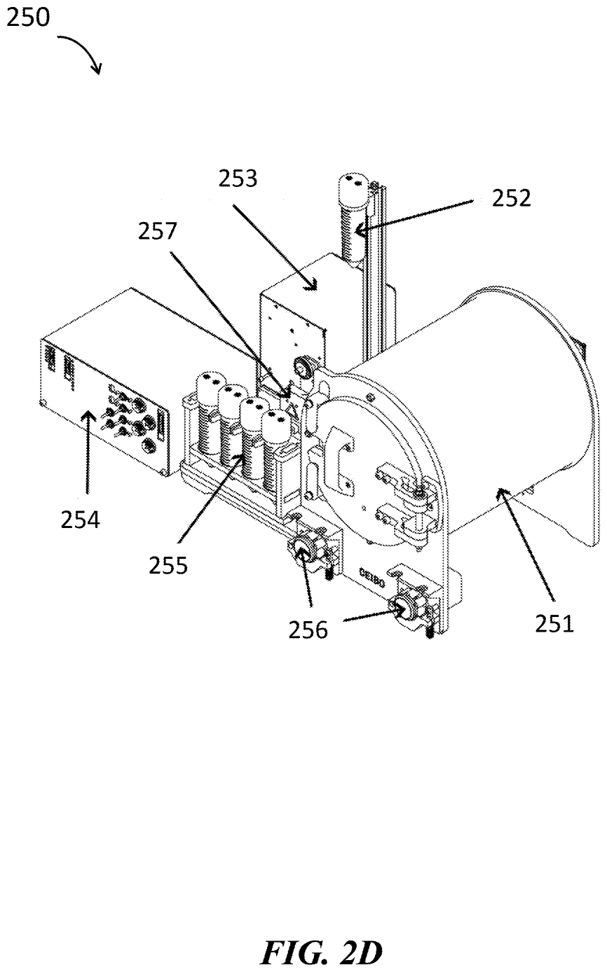

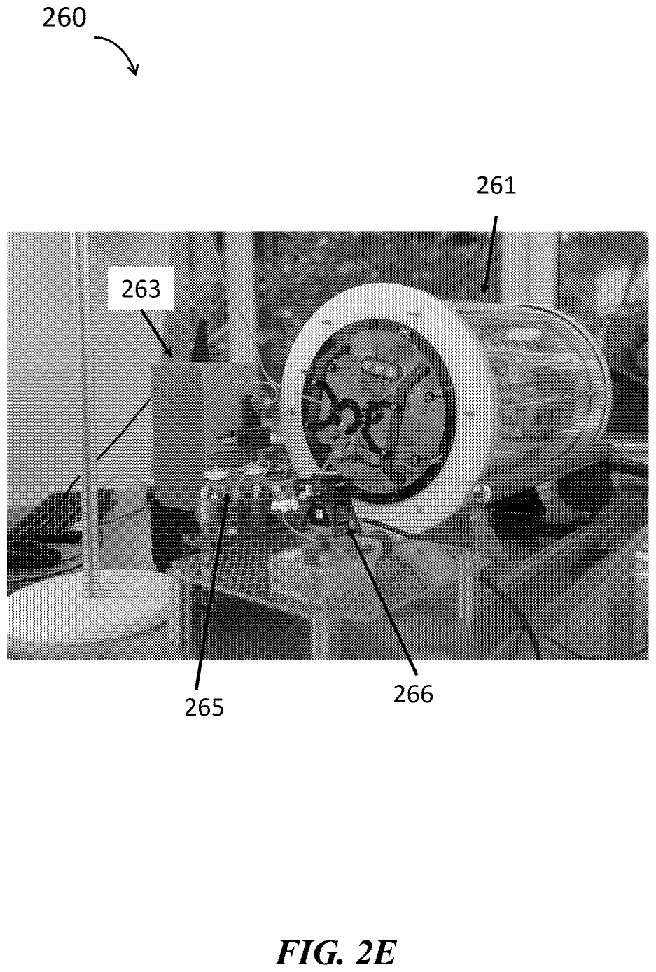

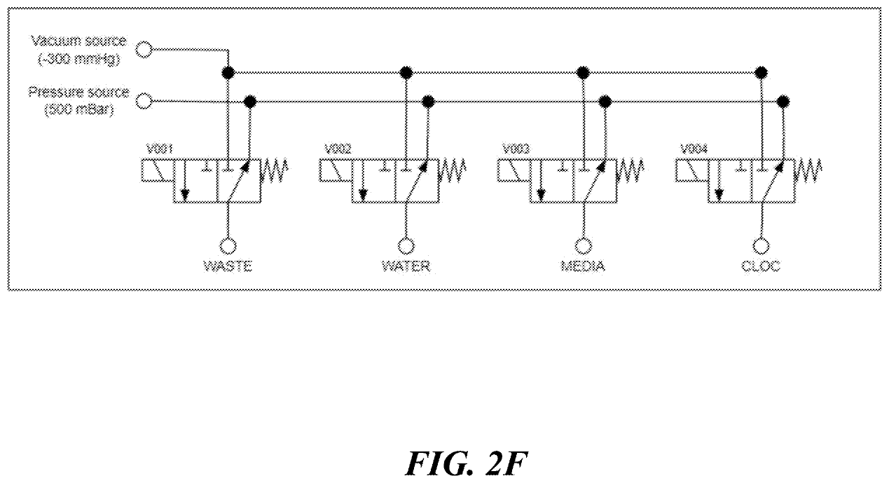

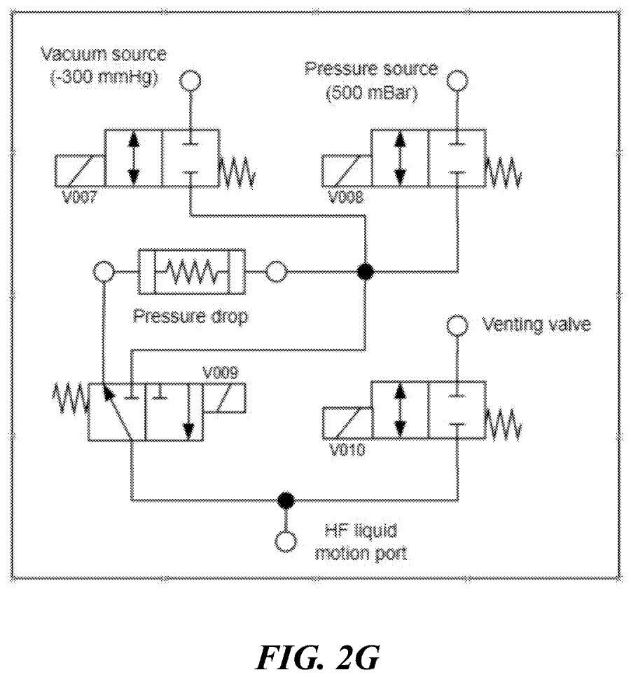

FIG. 2A shows an exemplary diagram of a cell culture system. The cell culture system 200 comprises a bioreactor 201, a flipper module 202, and a harvest and feed module 203. The bioreactor 201 can be encased in a production chamber (or chamber) 210. The flipper module 202 and the harvest and feed module 203 can be enclosed inside or outside of the chamber 210. The harvest and feed module 203 is operably connected to the bioreactor 201 to supply culture media and/or cells to the bioreactor 201. The harvest and feed module 203 may be connected to a culture media reservoir 205 and a water reservoir 204. The harvest and feed module 203 can be connected to a valve control manifold 211 and a pressure control manifold 212 (e.g., PMMA-PMMA bonded manifolds and Lee valves), which can connect to a vacuum pump 216 and a compressor 217. The vacuum pump 216 can be regulated by a pressure gauge 215. The compressor 217 can be regulated by a pressure gauge 218. The chamber 210 can be coupled to a CO₂ tube or container 213 through a CO₂ control valve 214. In some embodiments, the chamber 210 can be coupled to a O₂ tube or container through a O₂ control valve. In some embodiments, the cell culture system may comprise one or more sensors. In some embodiments, the chamber 210 can comprise a heater temperature sensor (e.g., a DS18B20 sensor), a temperature-humidity sensor (e.g., a DHT22, BME280 or TH-MB04S sensor), and/or a CO₂ sensor (e.g., an MHZ-16B NDIR or MH-410D CO₂ sensor). In some embodiments, the chamber 210 can comprise a humidifier to regulate or control the humidity of the interior environment. In some embodiments, the chamber 210 can comprise an electrical heater to regulate or control the temperature of the interior environment. In some embodiments, the chamber 210 can comprise a fan to assist in the regulating or control of temperature and/or humidity.

In some embodiments, the CO₂ tube or container 213 can be operably coupled to a plurality of chambers comprising the cell culture system. In some embodiments, the valve control manifold 211 and pressure control manifold 212 can be operably coupled to a plurality of chambers comprising the cell culture system.

In some embodiments, the production chamber may comprise an oxygen sensor. In some embodiments, the production chamber may comprise a control loop for oxygen. In some embodiments, the production chamber may comprise one or more optical density sensors. In some embodiments, the one or more optical density sensors may be configured to measure or detect a density of the cells or cell density (or concentration) inside the bioreactor. In some embodiments, the one or more optical density sensors may be configured to monitor a density of the cells or cell density inside the bioreactor. In some embodiments, one or more sensors may measure or detect the cell density when the cells and medium are fed into the bioreactor. In some embodiments, one or more sensors may measure or detect the cell density during the production of the cells (e.g., during the operation of the bioreactor). In some embodiments, one or more sensors may measure or detect the cell density at the end of cell production. In some embodiments, one or more sensors may comprise an alarm mechanism. In some embodiments, the alarm mechanism may send an alarm to the system or the bioreactor if a measurement exceeds a threshold value.

In some embodiments, the optical density sensor may measure a light intensity that passes through a sample. In some embodiments, the optical density sensor may comprise a light-emitting source (e.g., a light-emitting diode or LED light source) and a detector (e.g., a photodiode). In some embodiments, the optical density sensor may measure a reflection of light. In some embodiments, the optical density sensor may measure a transmission of light. The light intensity that passed through the sample may be correlated with a reference light intensity (e.g., a blank) using the Beer-Lambert law. A calibration curve for known concentrations may be constructed (e.g., by plotting light intensity vs. cell concentration). A cell concentration may be calculated or extrapolated from the calibration curve. FIG. 2B shows an exemplary three-point calibration curve using three different cell concentrations (e.g., 2×10⁶ cells/mL, 4×10⁶ cells/mL, and 8×10⁶ cells/mL), correlating the absorbance of the sample with its concentration. The calibration curve may be used to estimate cell concentrations based on the absorbance signal obtained. FIG. 2C shows exemplary concentration measurements by an optical density sensor disclosed in the present disclosure and a commercial device (e.g., Cell Counter). Multiple measurements were performed when the bioreactor was being washed. A sample was taken with the harvest and feed module every 10 mL and the cells were countered. FIG. 2C shows comparative total cell concentration, using optical density sensor and cell counter after passing through the harvest and feed module. The measurement by the optical density sensor has high agreement with the cell counter measurement.

FIG. 2D shows an exemplary cell culture system. The cell culture system 250 may comprise a chamber 251. The chamber 251 may have controlling mechanisms to control the environmental conditions of the chamber. The environmental conditions may comprise temperature, CO₂ content or concentration, O₂ content or concentration, and/or humidity. The chamber may comprise one or more bioreactors. The one or more bioreactors may comprise a bioreactor as disclosed herein. In some embodiments, the one or more bioreactors may comprise a different type of bioreactor. In some embodiments, the one or more bioreactors may comprise a bioreactor that has a single gyroid or double gyroid structure. In some embodiments, the one or more bioreactors may be coupled to one or more flippers. The flipper may be configured to flip the bioreactor to aid in mixing of cells and medium, diffusion of gases in the medium, and/or resuspension of cells. The chamber 251 may comprise one or more sensors as disclosed in the present disclosure. In some embodiments, the chamber may further comprise one or more actuators that allow for the control of one or more of the environmental conditions. In some embodiments, the one or more actuators may be configured to regulate or control one or more fans, gas intakes, or heaters. The cell culture system 250 may comprise one or more reservoirs (e.g., 255). The one or more reservoirs may be configured to store and supply one or more reagents (e.g., cell culture media, water, wash solutions, or buffers). The one or more reservoirs may be connected to a harvest and feed module. The one or more reservoirs may be connected to one or more pumps. The cell culture system 250 may comprise one or more elevated reservoirs (e.g., 252). The one or more elevated reservoirs may be configured to store or supply one or more reagents and the one or more elevated reservoirs may be elevated to create a pressure in the bioreactor. The pressure in the bioreactor may prevent the entry of air into the bioreactor. The cell culture system 250 may comprise a sampling platform 253. The sample platform may be configured to handle one or more reagents. The sampling platform may further be configured to extract one or more samples from the bioreactor for measurement. The sampling platform may comprise or be coupled to one or more sensors (e.g., optical density sensor 257). The sampling platform may allow for the handling of multiple fluids (e.g., cell culture, fresh media, water, etc.). The sampling platform may enable the measurement of cell concentration based on absorbance in an in-line manner without compromising sterility. The sampling platform may allow the sample to be returned to the bioreactor. In some embodiments, the sample may not be returned to the bioreactor. The cell culture system 250 may comprise one or more pump controllers (e.g., 254). The one or more pump controllers may control one or more pumps and/or valves (e.g., turning on/off, sequencing). The pump controller can operate various valve types to open or close fluid circulation pathways. The cell culture system 250 may comprise one or more pumps (e.g., 256). A pump may be configured to move the fluid based on a sequence determined by the user or a program. In some embodiments, the cell culture system 250 may comprise one or more peristaltic pumps. The one or more peristaltic pumps may be configured to move liquid within the cell culture system, enabling operation in various modes (e.g., re-injection, recirculation, continuous or alternating perfusion, and/or sample collection) at chosen flow rates for the required time. The pumps can operate with tubing of different diameters. The pumps may have one or more rollers, reducing flow pulsation. The location of the pumps may be flexible and determined by the user.

In some embodiments, the cell culture system may be considered a master or main module. The components of the cell culture system may be considered as sub-modules. Any changes in the operation or operation sequences can be implemented via the main module that commands the sub-modules. The desired sequence or operation may be uploaded to the main module. The desired sequence or operation may be transmitted to one or more sub-modules (e.g., the pump or pump controller). The commands from the main module may regulate or control the sub-modules to operate according to the commands.

FIG. 2E shows an image of a part of an exemplary cell culture system 260. The cell culture system 260 may comprise a chamber 261, a pump 266, one or more reservoirs 265, and a sampling platform 263. The cell culture system 260 may further comprise one or more sensors (not shown), one or more pump controllers (not shown), and one or more elevated media reservoirs (not shown).

FIG. 2F shows an exemplary valve control manifold. In some embodiments, the valve control manifold may comprise one or more controllers connected to one or more reservoirs (e.g., water, media) and/or bioreactors. The one or more controllers may be connected to a vacuum source (e.g., 300 mmHg) and a pressure source (e.g., 500 mBar). In some embodiments, the one or more controllers may regulate the connection of the one or more reservoirs and/or bioreactors to the vacuum source or pressure source. In some embodiments, the one or more controllers may comprise a switch to change between the connection to a vacuum source or a pressure source. FIG. 2G shows an exemplary pressure control manifold. In some embodiments, the pressure control manifold may comprise one or more controllers connected to a vacuum source or a pressure source. In some embodiments, the one or more controllers may be configured to connect the manifold to a vacuum source or a pressure source. In some embodiments, the pressure control manifold may comprise one or more pressure drop modules. In some embodiments, the pressure control manifold may comprise one or more venting valves configured to vent the pressure. In some embodiments, the pressure control manifold may be connected to the H&F liquid motion port. In some embodiments, the pressure control manifold may be connected to the bioreactor. In some embodiments, the pressure control manifold may be connected to a chamber of the cell culture system.

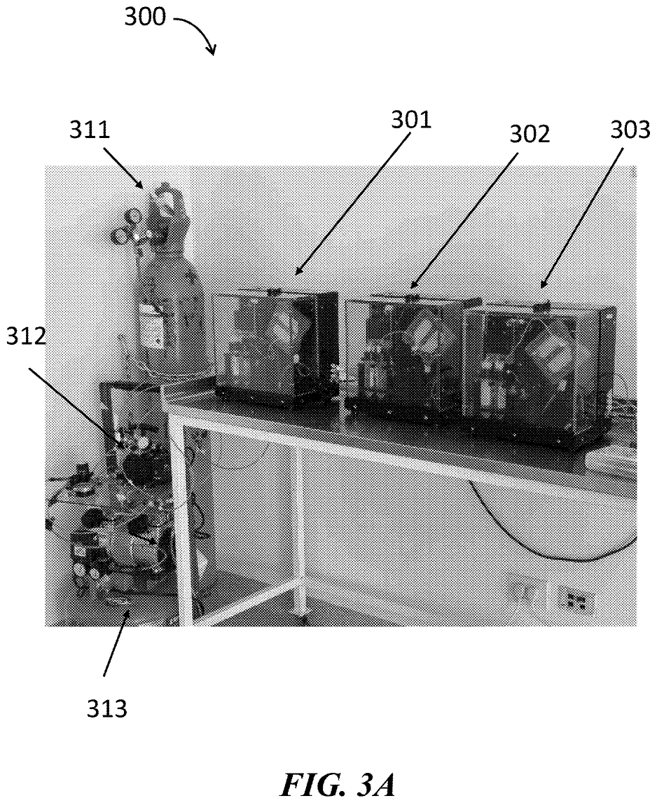

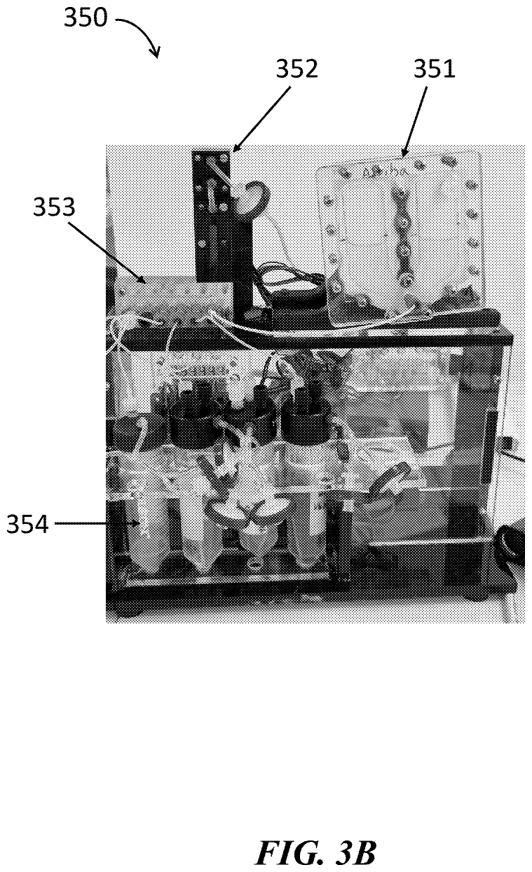

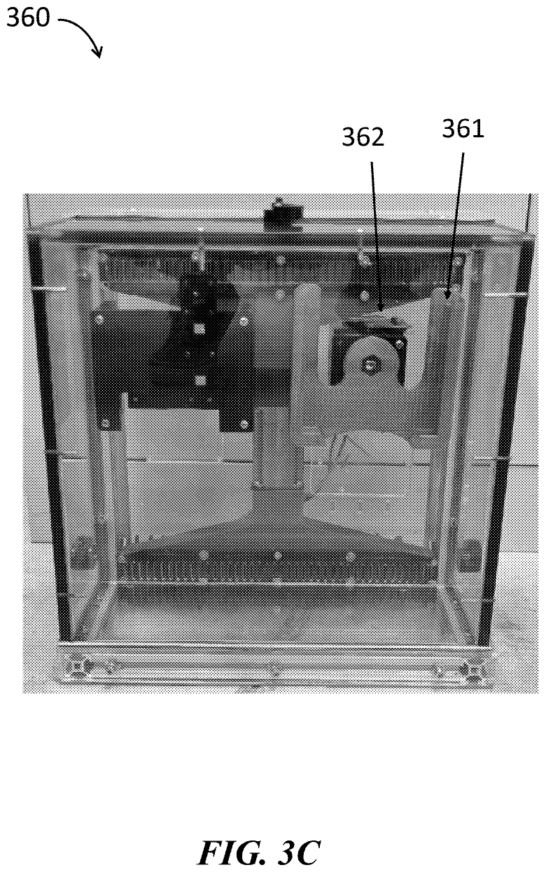

FIG. 3A shows an exemplary configuration of cell culture systems with external CO₂ tank, compressor, and vacuum pump. Three cell culture systems (e.g., chambers 301, 302, and 303) are operably connected to a CO₂ tank 311, a vacuum pump 312, and a compressor 313. FIG. 3B shows an image of a part of an exemplary cell culture system. The cell culture system 350 may comprise a bioreactor 351, a harvest and feed module 352, a manifold 353 (configured to regulate or control the fluids), and one or more reservoirs (e.g., 354). FIG. 3C shows an image of a chamber of an exemplary cell culture system. The chamber 360 may comprise a loading module 361 configured to hold or secure a bioreactor. The loading module may be coupled to a flipper module 362 configured to flip the bioreactor.

In some embodiments, the cell culture system may be configured for cell line growth. In some embodiments, the bioreactor provided herein can be considered as a platform for cell line on a chip (CLOC) operation. In some embodiments, the cell culture system may be configured to inoculate a cell or a plurality of cell. In some embodiments, the cell culture system may be configured to control an environmental condition or parameter. In some embodiments, the cell culture system may be used to grow cells. In some embodiments, the cell culture system may be configured to grow cells at maximum cell density. In some embodiments, the cell culture system may be configured to grow cells to an optimized cell density (e.g., for cell production or biological material production). In some embodiments, the cell culture system may be configured to maintain a cell density (e.g., for cell production or biological material production). In some embodiments, the cell culture system may be configured to produce one or more biological materials. In some embodiments, the one or more biological materials may comprise a protein, a peptide, or a polypeptide. In some embodiments, the one or more biological materials may comprise one or more antibodies. In some embodiments, the one or more biological materials may comprise one or more drugs.

In some embodiments, a bioreactor may be loaded inside the production chamber. In some embodiments, the bioreactor may be connected via a cable to the communication BUS or communication module. Once powered on, the bioreactor may be allowed to initialize so it is ready to receive and run a command. In some embodiments, a new bioreactor may be configured as a client of the main module (e.g., the cell culture system). In some embodiments, the cell culture system may have one or more inputs. In some embodiments, the one or more inputs may comprise a primary input for use as a master for data storage and communication. In some embodiments, the one or more inputs may comprise a secondary input for communication as a client.

In some embodiments, the cell culture system can be operated in a continuous mode. In some embodiments, the cell culture system can be operated in a batch mode. In some embodiments, the cell culture system can be operated in a semi-batch mode.

In some embodiments, the cell culture system provided herein can be used for the optimization of conditions for cell growth in liquid culture that can then be applied to growth in the bioreactor for cell and/or product (e.g., antibody) production. In some embodiments, the cell culture system provided herein can be used to test culture conditions or media, for example using multiple conditions with multiple bioreactors. In some embodiments, the cell culture system provided herein can be used as a continuous feeding system for a bioprocessor.

In some embodiments, the cell culture system may be configured to inoculate cells. In some embodiments, the cell culture system may be configured to inoculate cells to a desired concentration. In some embodiments, the cell culture system may be configured to a desired lifecycle stage. In some embodiments, the cell culture system may be configured to provide cell cultures to a larger scale bioprocessor. In some embodiments, the cell culture system may be configured to generate a continuous feed of cells at a constant concentration (e.g., 0.1×10⁶ cells/mL to 1×10⁷ cells/mL) to a bioprocessor.

### Flipper Module

A bioreactor disclosed herein can be flippable about a horizontal axis. In some embodiments, the bioreactor is operably coupled to a flipper module to modulate the rotation, rocking, or flipping of the bioreactor. A flipper module can comprise a motor driving the rotation, rocking, or flipping of the bioreactor, at least one barrier, and at least one sensor.

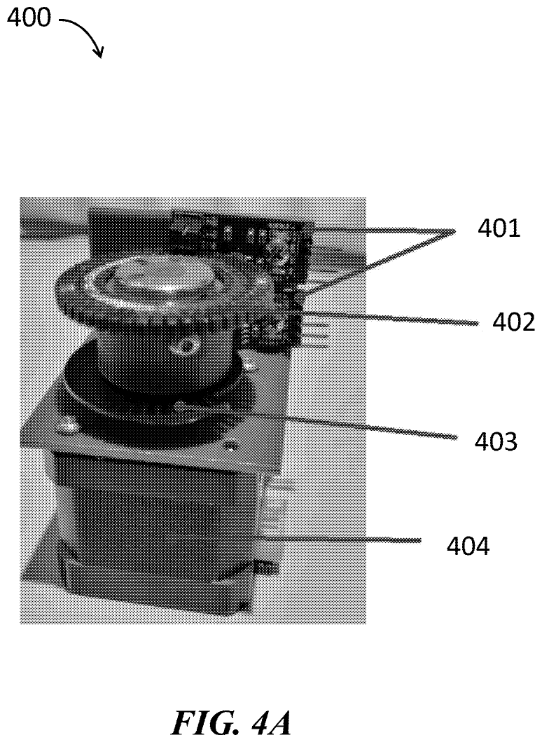

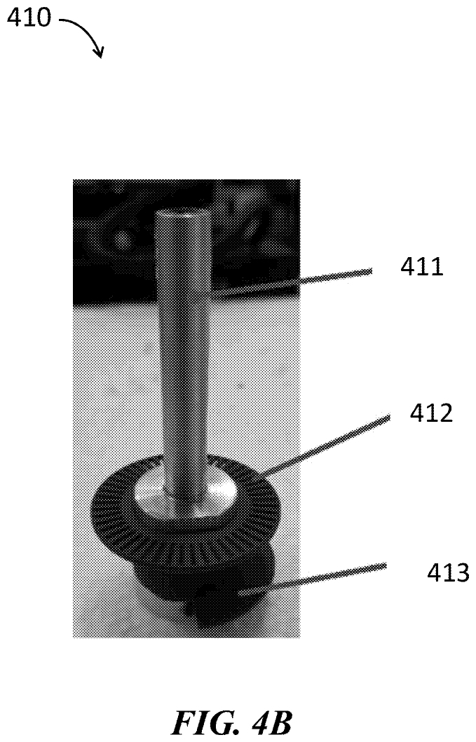

FIG. 4A shows an example flipper module. The flipper module 400 comprises a NEMA17 motor 404, a half-moon barrier 403, an additive barrier 402, and a plurality of sensors 401.

In some embodiments, the half-moon barrier can be configured for the sensor to detect the position and/or the movement of the rotor and/or the bioreactor. For example, when the slit of the half-moon barrier lets the light pass through, this indicates the device is at 0° or inverted at 180°. The half-moon barrier can serve as an imposed reference. In some embodiments, the additive barrier can be configured for the sensor to detect the position and/or the movement of the rotor and/or the bioreactor. For example, when the slits of the additive barrier let the light pass through, this indicates a fraction of an angle is added to the trajectory the device has moved. This can further help decide whether the bioreactor is rotated to the predefined positions. The combination of the half-moon barrier and additive barrier can provide information about the position of the bioreactor. If the position does not match the pre-defined position, an alarm may be triggered to notify the user.

In some embodiments, the plurality of sensors can detect and verify the connection between the flipper module and the bioreactor, the position of the bioreactor, and/or the angular speed of the rotation, and/or the angular acceleration of the rotation.

FIG. 4B shows example components of a flipper module. The flipper module comprises a shaft axis 411, an additive barrier 412, and a half-moon barrier 413.

In some embodiments, the shaft axis 411 is configured to be in the horizontal direction, therefore rotating or flipping the bioreactor about a substantially horizontal axis. When the bioreactor is rotated about a horizontal axis, the rotational centripetal forces developed in the culture medium are in part dependent from the dimension of the bioreactor and the speed of rotation. The dimension of the bioreactor and the speed of rotation can be optimized such that the cells are not distributed to the outer perimeter of the bioreactor and the rotation is sufficient to maintain the cells in the culture medium.

### Harvest and Feed Module

The cell culture system can comprise a harvest and feed module configured to supply culture medium and/or cells to the bioreactor and/or to remove cells, media and/or other substances from the bioreactor.

In some embodiments, the harvest and feed module can modulate the volume and speed of the culture medium and/or cells that are supplied into the bioreactor and/or the volume and speed of cells, media and/or other substances from the bioreactor.

The cell culture system can comprise one or more reservoirs for holding the liquid before supplying it to the harvest and feed module. In some embodiments, the system includes at least 1, at least 2, at least 3, at least 4, at least 5, at least 6, at least 7, at least 8, at least 9, at least 10, or more reservoirs. The reservoirs can be filled asynchronously, so that one reservoir fills while another, already fully filled, is used to supply the harvest and feed module with liquid. In some embodiments, the volume of the reservoir may be linked to throughput of the bioreactor. In some embodiments, multiple reservoirs may be installed in parallel and uncoupled from each other. In some embodiments, multiple reservoirs may be installed in series.

The harvest and feed module can comprise one or more sensors. The sensors can measure parameters including pH and/or temperature of the medium. A sensor may be an in-line sensor or may be connected to a sampling device that samples media or cells intermittently from one or more components of the harvest and feed module. The harvest and feed module can provide media and/or cells at a range of rates dependent on the use, scale and operation of the system. In some embodiments, the harvest and feed module can provide from about 1 microliter (μL) to about 1 liter (L) of liquid media (or cells in a liquid) per hour to the bioreactor.

The harvest and feed module may be coupled to one or more pumps for flowing liquid from a reservoir to the harvest and feed module. The system may include greater than or equal to 1, 2, 3, 4, 6, 8, 10, or more pumps. The pumps may be the same type of pump or may be different types of pumps. Example pumps include a syringe pump, a peristaltic pump, and a pressure pump. In some embodiments, the harvest and feed module may be configured to provide unidirectional flow through to the reactor. The pumps may work synchronously or individually. The one or more pumps supply liquid with a high degree volume and rate accuracy to the harvest and feed module. In some embodiments, the accuracy is within 1, 2, 3, 4 or 5 nanoliters. In some embodiments, the harvest and feed module can be connected to a pressure gauge.

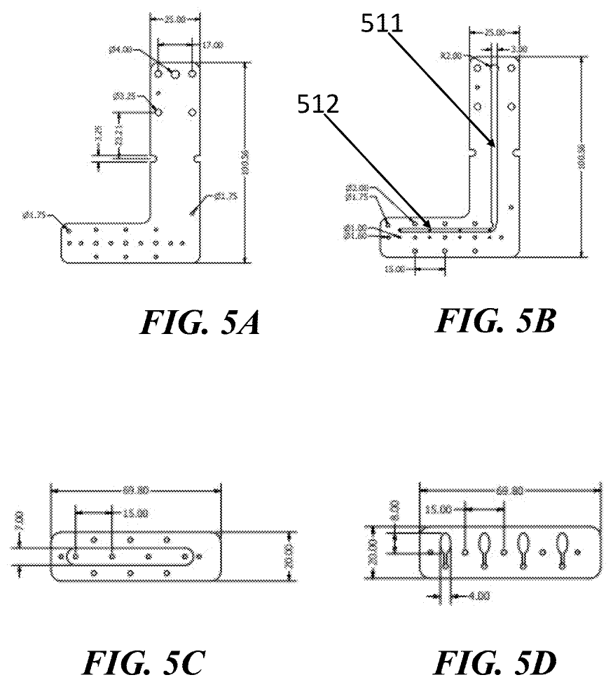

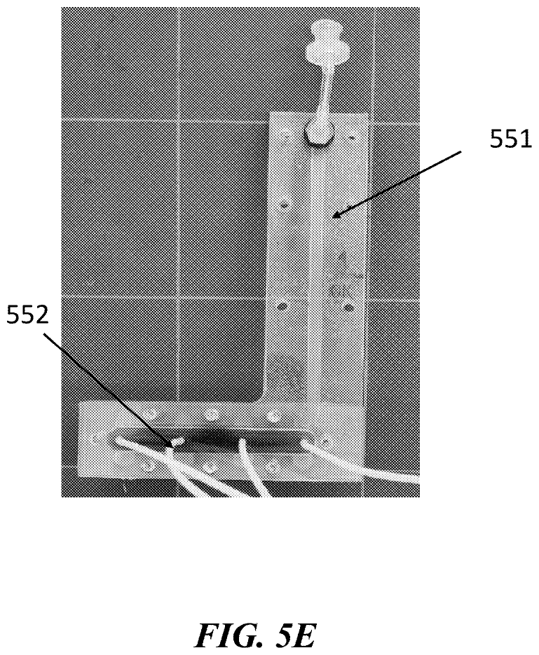

FIGS. 5A-5D show components of an example harvest and feed module. FIG. 5A shows the harvest and feed module can have a L-shape and can comprise a plurality of holes for connecting to other components of the harvest and feed module. FIG. 5B shows an internal configuration of the harvest and feed module. The harvest and feed module can comprise one or more microchannels 511 and holes 512 for providing passage from the tubing to the microchannels. FIG. 5C shows a tubing support of the harvest and feed module. FIG. 5D shows a valve cap of the harvest and feed module.

FIG. 5E shows an example harvest and feed module that is assembled from components of FIGS. 5A-5D. The harvest and feed module comprises microchannels 551 and is connected to external device or reservoir through tubing 552.

### Firmware and Applications

The cell culture system can comprise a plurality of applications that can be used with and control the bioreactor, the flipper, and/or the harvest and feed module.

In some embodiments, the cell culture system can comprise a user interface (UI) for the operation and/or control of the cell culture system.

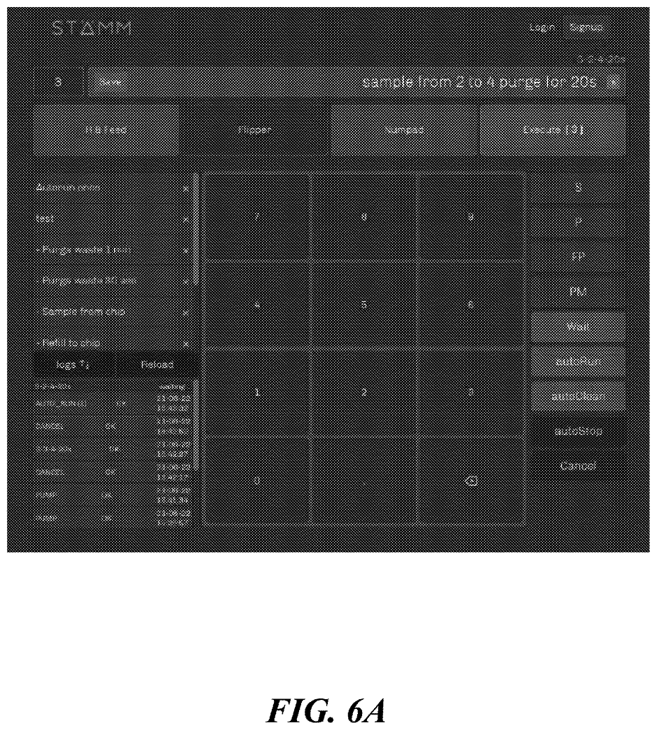

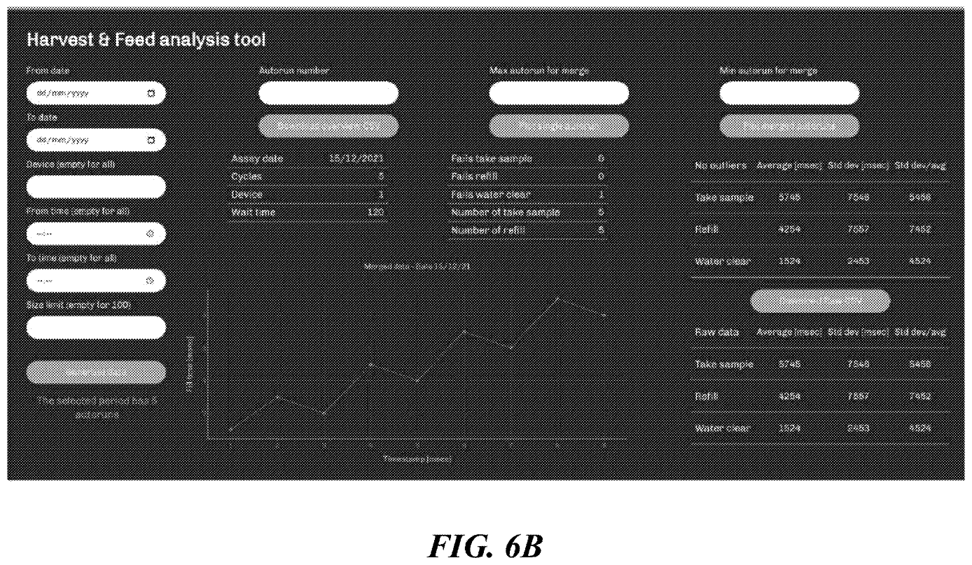

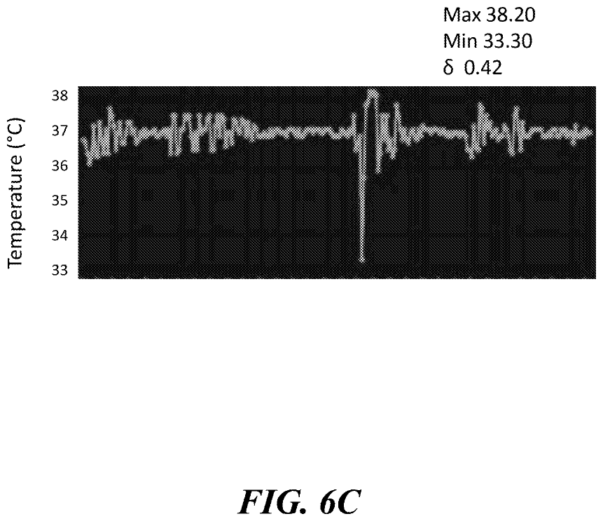

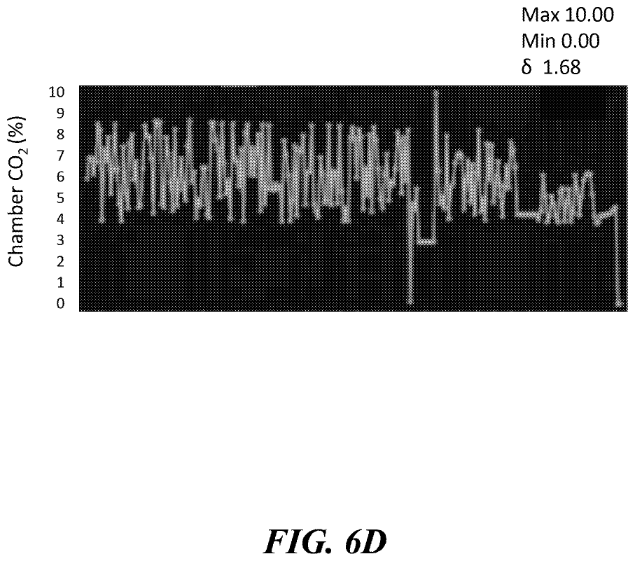

FIG. 6A shows an example screen of the user interface. In the UI, the user can elect for operation of harvest and feed (H&F) or the flipper. The UI also comprises a number pad (Numpad) to input the numerical parameters. The UI can program the operation process. The electable conditions or operations can comprise autorun, autoclean, autostop, purge, sample, refill, pump, test, cancel, execute, etc.

In some embodiments, the applications can comprise a harvest and feed (H&F) analysis tool configured to control and monitor the feeding volume, feeding rate, and/or pressure of the harvest and feed module to the bioreactor. FIG. 6B shows an example screen of the H&F analysis tool. In some embodiments, the H&F analysis tool may be configured to search and analyze H&F autoruns. In some embodiments, the H&F analysis tool may be configured to perform operation time analytics, media and standard deviation analysis. In some embodiments, the H&F analysis tool may be configured to analyze errors. In some embodiments, the H&F analysis tool may comprise modules for inputting autorun information and sampling sequence (e.g., intervals for sampling, refill, and water wash).

In some embodiments, the applications can comprise a datalogger that is configured to visualize real time data of the sensor and actuators. Data can also be downloaded from the datalogger for analysis with other tools. FIGS. 6C and 6D show exemplary temperature and CO₂ data during the operation of the bioreactor. In some embodiments, the datalogger may reveal real time sensor values. In some embodiments, the datalogger may be configured to show historical data of an assay. In some embodiments, the datalogger may be configured to show basic analytics of the data.

In some embodiments, the application can comprise a scheduler configured to schedule multiple tasks or routines at any time. In some embodiments, the scheduler may be configured to execute saved presets, commands, or instructions on controllers at desired time point (e.g., date and time).

In some embodiments, the cell culture system may comprise a communication interface that allows for transmitting data of the cell culture system. The data may be transmitted to an electronic device in communication with the communication interface. The communication interface may be a wireless communication interface, a Wi-Fi interface, a near-field communication interface, or a Bluetooth interface, as described herein. The electronic device may be a device that may communicate with the communication interface, e.g., a mobile device (e.g., smart phone, tablet, laptop, etc.). Alternatively, the communication interface may be a wired communication interface.

The cell culture system can comprise a plurality of firmware to command and control the multiple actions the bioreactor can execute. The multiple actions can comprise sensor reading, message queuing telemetry transport (MQTT) communication, Wi-Fi or serial connectivity, actuation of the components, and alarming system.

### Method of Growing Cells

In an aspect, the present disclosure provides a method of growing cells, comprising: (a) inoculating a bioreactor disclosed herein with a liquid medium and at least one cell, and (b) incubating the bioreactor for a period of time under conditions sufficient to permitting cell growth (e.g., cell division and replication); wherein during the incubating, rotating the bioreactor for a pre-determined angle at pre-determined time periods.

In some embodiments, the rotating is along a horizontal axis.

In some embodiments, the rotating is performed following a pre-determined sequence. Alternatively or in addition to, the rotating is performed at a constant annular speed.

In some embodiments, the sequence can comprise (i) rotating the bioreactor by a first degree at a first speed; (ii) rotating the bioreactor by a second degree at a second speed; (iii) rotating the bioreactor by a third degree at a third speed; and (iv) repeating steps (i)-(iii).

In some embodiments, the first degree, the second degree, and the third degree are same. In some embodiments, at least two of the first degree, the second degree, and the third degree are different. In some embodiments, the first degree, the second degree, and/or the third degree is from about 60 degrees (°) to about 90°, from about 60° to about 120°, from about 60° to about 150°, from about 60° to about 180°, from about 90° to about 120°, from about 90° to about 150°, from about 90° to about 180°, from about 120° to about 150°, from about 120° to about 180°, or from about 150° to about 180°. In some embodiments, the first degree, the second degree, and/or the third degree is about 60°, about 90°, about 120°, about 150°, or about 180°.

In some embodiments, the first speed, the second speed, and the third speed are same. In some embodiments, at least two of the first speed, the second speed, and the third speed are different. In some embodiments, the first speed, the second speed, and/or the third speed is from about 10°/s to about 15°/s, from about 10°/s to about 30°/s, from about 10°/s to about 60°/s, from about 10°/s to about 90°/s, from about 15°/s to about 30°/s, from about 15°/s to about 60°/s, from about 15°/s to about 90°/s, from about 30°/s to about 60°/s, from about 30°/s to about 90°/s, or from about 60°/s to about 90°/s.

In some embodiments, subsequent to (i), (ii), and/or (iii), the bioreactor is held at the position for a holding time. In some embodiments, the holding time is from about 1 s to about 2 s, from about 1 s to about 3 s, from about 1 s to about 4 s, from about 1 s to about 5 s, from about 1 s to about 10 s, from about 1 s to about 20 s, from about 1 s to about 30 s, from about 1 s to about 1 minute (min), from about 2 s to about 3 s, from about 2 s to about 4 s, from about 2 s to about 5 s, from about 2 s to about 10 s, from about 2 s to about 20 s, from about 2 s to about 30 s, from about 2 s to about 1 min, from about 5 s to about 10 s, from about 5 s to about 20 s, from about 5 s to about 30 s, from about 5 s to about 1 min, from about 10 s to about 20 s, from about 10 s to about 30 s, from about 10 s to about 1 min, from about 20 s to about 30 s, from about 20 s to about 1 min, or from about 30 s to about 1 min.

In some embodiments, the rotating comprises (i) rotating the bioreactor by about 180° at a speed of about 30°/s and holding the bioreactor for 4 s, (ii) rotating the bioreactor back to the original position at a speed of about 30°/s in an opposite direction or a same direction and holding the bioreactor for 4 s, (iii) rotating the bioreactor at a speed of about 30°/s by about 180° in an opposite direction or a same direction and holding the bioreactor for 4 s, (iv) rotating the bioreactor back at a speed of about 30°/s to the original position in an opposite direction or a same direction and holding the bioreactor for 4 s, and (v) repeating (i)-(iv).

In some embodiments, the rotating comprises (i) rotating the bioreactor by about 180° at a speed of about 60°/s and holding the bioreactor for 4 s, (ii) rotating the bioreactor back to the original position at a speed of about 60°/s in an opposite direction or a same direction and holding the bioreactor for 4 s, (iii) rotating the bioreactor at a speed of about 60°/s by about 180° in an opposite direction or a same direction and holding the bioreactor for 4 s, (iv) rotating the bioreactor back at a speed of about 60°/s to the original position in an opposite direction or a same direction and holding the bioreactor for 4 s, and (v) repeating (i)-(iv).

In some embodiments, the rotating comprises (i) rotating the bioreactor by about 180° at a speed of about 15°/s and holding the bioreactor for 4 s, (ii) rotating the bioreactor back to the original position at a speed of about 15°/s in an opposite direction or a same direction and holding the bioreactor for 4 s, (iii) rotating the bioreactor at a speed of about 15°/s by about 180° in an opposite direction or a same direction and holding the bioreactor for 4 s, (iv) rotating the bioreactor back at a speed of about 15°/s to the original position in an opposite direction or a same direction and holding the bioreactor for 4 s, and (v) repeating (i)-(iv).

In some embodiments, the cell medium is at most 90%, at most 89%, at most 88%, at most 87%, at most 86%, at most 85%, at most 84%, at most 83%, at most 82%, at most 81%, or at most 80% volume of the cell culture chamber. In some embodiments, the cell medium is at least 50%, at least 55%, at least 60%, at least 65%, at least 70%, at least 75%, at least 80%, or at least 85% volume of the cell culture chamber, including increments therein. In some embodiments, the cell medium is about 80% volume of the cell culture chamber.

In some embodiments, the bioreactor is incubated for a period of time from 1 day to 10 days, from 1 day to 20 days, from 1 day to 30 days, from 1 day to 40 days, from 1 day to 50 days, from 1 day to 100 days, from 10 days to 20 days, from 10 days to 30 days, from 10 days to 40 days, from 10 days to 50 days, from 10 days to 100 days, from 20 days to 30 days, from 20 days to 40 days, from 20 days to 50 days, from 20 days to 100 days, from 30 days to 40 days, from 30 days to 50 days, from 30 days to 100 days, from 40 days to 50 days, from 40 days to 100 days, or from 40 days to 100 days.

In some embodiments, an in-line sensor can be configured to determine a cell density. Alternatively or in addition to, a sample can be taken or drawn from the bioreactor to determine a cell density.

In some embodiments, the cells can be harvested after culturing in the bioreactor continuously, in a batch mode, or in a semi-batch mode. In some embodiments, a substance produced by the cells, such as a protein or antibody can be harvested after culturing in the bioreactor continuously, in a batch mode, or in a semi-batch mode. In some embodiments, when the sensor senses a certain cell density (e.g., a predetermined cell density), the cells or a substance produced by the cells can be harvested.

### Computer Systems

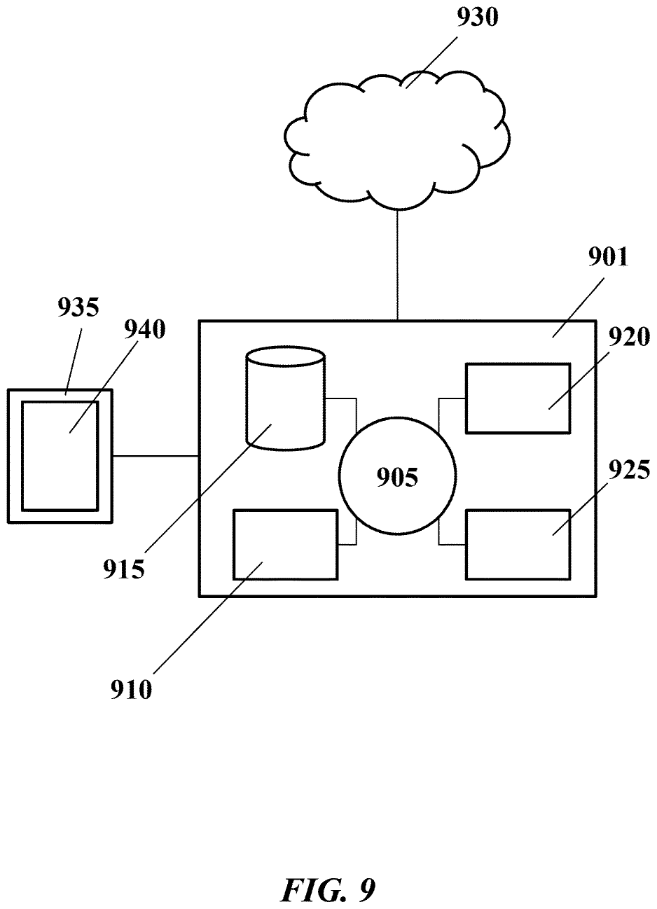

The present disclosure provides computer systems that are programmed to implement methods of the disclosure. FIG. 9 shows a computer system 901 that is programmed or otherwise configured to control all the internal processes of the systems as programmed, such as data acquisition through embedded sensors (e.g., physical, chemical, and biological data), sensor data fusion and commanding control loops, image processing, and creating data sets associated with each process. The computer system 901 can regulate various aspects of the production process. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user (e.g., Virtual Private Networks, Computer hosted in services such as Amazon Web Services (AWS), Satellite communication). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, settings, bioprocess report listing measured variables in real time of every stage of the system, capabilities to export and import files (e.g., configuration files, updates), calibration, alarms (e.g., errors, maintenance, replacement of consumables). Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, adjust variables of the control systems using feedback loops, detect problems in the process by image recognition and pattern analysis, fuzzy logic and with hard and soft threshold enforcements, correlate specific and unspecific data through machine learning (e.g., Supervised, Unsupervised, and/or Reinforcement) to optimize process conditions within the system, the process outcomes, modelling behavior and simulation.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Example 1: Cell Culture System for Antibody (Ab) Production

The cell culture system consists of the following modules: (1) cell stocking module comprising a U-shaped bioreactor, (2) cell production module containing an additional bioreactor (e.g., a gyroid bioreactor), (3) harvest and feed module, and (4) storage module. The cell culture system can be a standalone system which can be installed at the manufacturing site. Each module permits swappable components, so that components are readily swapped in and out. The bioreactor acting as a cartridge in the cell culture system can be rapidly installed, started and run for a fixed length of time, then readily swapped out for a new run.

Cell culture system inputs: cell bank for cells which produce desired protein product (e.g., antibody), culture medium, water, glucose, CO₂ and O₂.

Cell culture system outputs: cells, protein product (e.g., antibody), and metabolic by-products. Output includes measurements of cell density and cell viability.

The cell culture system was maintained at 37° C. with fixed concentrations of CO₂ and O₂. Sensors placed throughout the system include O₂, glucose, temperature, and pH sensors.

The cell stocking module comprising the U-shaped bioreactor was configured to generate a continuous feed of cells at a constant concentration 1×10⁶ cells/mL.

Cells were inoculated from the cell bank. Number of cell divisions/passages were calculated such that the bioreactor maintained a cell line until a pre-determined number of passages was achieved. Cells were inoculated in the U-shaped bioreactor to a concentration of 1×10⁶ cells/mL and were fed continuously from the cell stocking module to the cell production module for growth of cells and production of antibodies.

The bioreactors (e.g., U-shaped bioreactor and gyroid reactor) were maintained at 37° C. with fixed concentrations of CO₂ and humidity.

The harvest and feed module was maintained at 4° C. to prevent degradation of cell and antibody/protein products.

The storage module was maintained at 4° C. and was used to store the culture media.

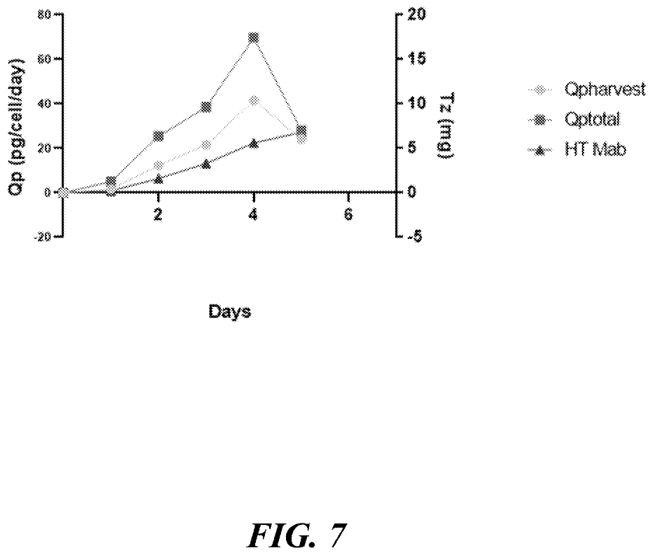

FIG. 7 shows a graph of specific total productivity (Qptotal), specific harvestable productivity (Qpharvest) and accumulated monoclonal antibody (mAb, or high throughput monoclonal antibody, “HT Mab”) Tz produced by the cells. The harvestable specific productivity averaged 20.3 μg/cell/mL. The maximum value of total specific productivity was 67.88 μg/cell/mL with a cell concentration of 3.5×10⁶ cells/mL. In a 5-day process, 7 mg of accumulated mAb was produced with only 90 mL of media used.

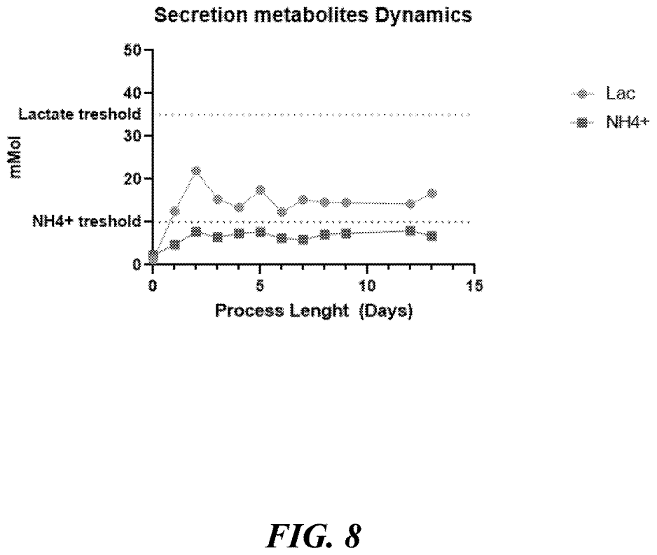

FIG. 8 shows the secretion metabolite dynamics of the cell culture grown in the bioreactor. The cell culture system had low toxic metabolite concentration (Lactateₘₐₓ was about 20 mM and Amoniaₘₐₓ was about 7 mM).

The cell culture system operated in continuous mode had improved mAb quality compared with batch processes where cells can age inside the bioreactor, making the later days of production of a lower quality. Table 1 shows antibody production run values and projections.

TABLE 1

| | | | | | | | 50 × 20 L |
|---|---|---|---|---|---|---|---|
| | | | | Projected | Projected | | to generate |
| | | Projection | Projection | 1 L | 10 L | CDMO | 45 kg |
| | Run values | 1 | 2 | bioreactor | bioreactor | Brx 20 L | Ab/quarter |
| Bioreactor | 0.008 | 0.066 | 0.15 | 1 | 10 | 20 | 1000 |
| Volume (L) | | | | | | | |
| Media Usage (L) | 0.075 | 0.1 | 2.25 | 90 | 900 | 1800 | 90000 |
| Qpavg | 10-60 | 50 | 50 | 50 | 50 | 50 | 50 |
| (pg/cell/day) | | | | | | | |
| Cell Density | 2 × 10⁵-2 × 10⁷ | 1 × 10⁷ | 1 × 10⁷ | 1 × 10⁷ | 1 × 10⁷ | 1 × 10⁷ | 1 × 10⁷ |
| (cell/mL) | | | | | | | |
| Ab Concentration | 0.1-1 | 0.5 | 0.5 | 0.5 | 0.5 | 0.5 | 0.5 |
| (mg/mL) | | | | | | | |
| Processor run time | 10-20 | 5 | 15 | 90 | 90 | 90 | 90 |
| (days) | | | | | | | |
| Accumulated mAb | 0.01-0.5 | 0.165 | 1.125 | 45 | 450 | 900 | 45000 |
| production (g) | | | | | | | |

### Example 2: Cell Growth in a Cell Culture System

This example demonstrates the functioning of the bioreactor and cell culture system disclosed in the present disclosure. A maximum cell density of 8×10⁶ cells/mL was achieved and maintained for at least 10 days. Growth of Chinese Hamster Ovary (CHO) cells was achieved, and the cells produced trastuzumab.

Samples with CHO cells in suspension were prepared. The samples had cell density of 1.23×10⁶ cells/mL and cell viability of 98.5%.

Three bioreactors (CMB4, CMB5, CMB6) were placed in three cell culture systems (Ceibo4, Ceibo5, Ceibo6) respectively. A first set of control tests with three CMB bioreactors (CMB1, CMB2, CMB3) was carried out in a commercial Numak incubator (37° C., 5% CO₂, 95% humidity). A second set of control tests was carried out in three 50 mL Erlenmeyer glass flasks with orbital agitation on a shaker in the incubator. The tests were performed in a biosafety cabinet in a clean room. All cell cultures were maintained at 37° C. and 5% CO₂. Each sample comprised 8 mL suspension with cell density of 1×10⁶ cells/mL in SCUP002 cell culture medium supplemented with antibiotic-antimycotic (ATB). The volume in all systems and control tests were maintained at 8 mL. Culture medium supplemented with ATB was changed every three days in the CMBs by emptying the culture medium reservoir using harvest and feed module in pump mode and aseptically refilling with fresh medium. The refill of the culture medium was performed with a 10 ml syringe prepared in the biosafety cabinet. After adding the medium, air was injected to push out residual volume from the reservoir.

A flipper and controller was set to operate the CMB in the following sequence: pause at 0° for 4 seconds, rotate 180° in 6 seconds, pause at 180° for 4 seconds, rotate 180° in 6 seconds, pause at 360° for 4 seconds, rotate −180° in 6 seconds, pause at 180° for 4 seconds, rotate −180° in 6 seconds, and pause at 0° for 4 seconds.

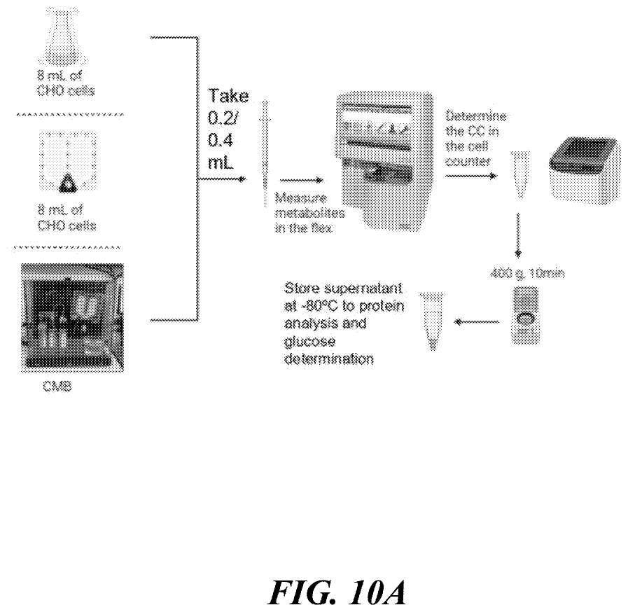

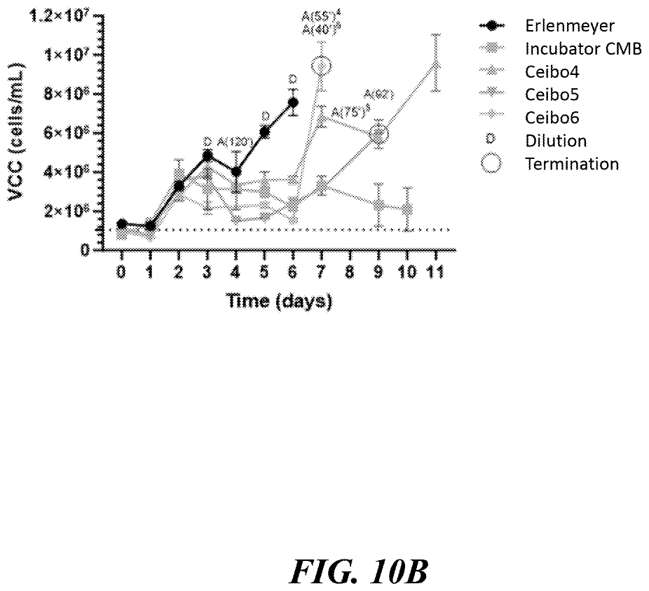

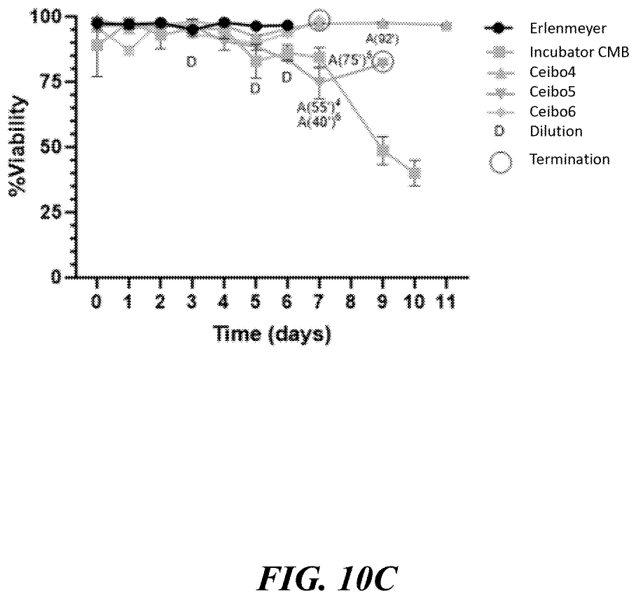

FIG. 10A shows an exemplary sampling scheme. Starting from day 0 and every 48 hours, 400 μL or 2 harvest and feed volumes of sample from the Erlenmeyer glass flasks or CMBs was collected. These samples were tested for cell concentration, cell viability, glucose, and trastuzumab (T_{z}) production. Metabolites may be measured with a FLEX system. Cell concentration may be measured by optical density sensor or cell counter. After the cell density measurement, the sample can be centrifuged (e.g., at 400 g speed for 10 min) to sediment the cells. The supernatant can be stored at −80° C. The supernatant may be subject to protein analysis and glucose determination. Trastuzumab may be measured by densitometry or spectrometry. The sampled volume was replenished with fresh medium. On other test days, 200 μL or 1 harvest and feed volume of sample was taken to measure cell concentration and cell viability. The sampled volume was replenished with fresh medium. The first stage of the test continued until a maximum cell density (DM) of 8×10⁶ cells/mL with over 90% viability was achieved. Once DM was reached, autorun sequences in the Ceibo system began, and the control cultures were manually diluted to match the final concentration of the Ceibo cultures for at least 10 days.

To sample from the Ceibo system, the following sequence of commands was loaded on the computer communicating with the 3 Ceibo systems: 1. check which CMB is communicating; 2. self-calibration; 3. purge the hose to waste for 5 seconds; 4. move the bioreactor to 0° to take the sample; 5. take from the bioreactor, send to waste, and purges for 15 seconds; 6. repeat 5 for 2 volumes samples; 7. move the bioreactor to 180° to replenish the sampled volume; 8. take from medium, send to bioreactor, and purge for 15 second; 9. repeat 8 for 2 volumes samples; 10. use harvest and feed module to release pressure inside the bioreactor; and 11. resume bioreactor flipping sequence.

Throughout the test, the Ceibo systems demonstrated good overall performance, maintaining the desired environmental conditions (humidity, temperature, and CO₂) and correctly performing the requested autoruns.

FIG. 10B shows average viable cell concentrations (VCC) over time (days) of CHO cells for Ceibo systems and control tests: Erlenmeyer controls in an incubator (control, solid circles), incubator CMBs (solid squares), and Ceibos (Ceibo4 triangles, Ceibo5 upside down triangles, and Ceibo6 diamonds). Open circles indicate hose entanglement events that caused a setup termination; D marks days where control dilutions were performed. A(X)^{Y} indicates the start of autorun in Ceibos, with X as the wait time in minutes between each autorun and Y as the Ceibo unit configuration. The dotted line represents the inoculum's cell concentration.

All systems developed similarly in terms of VCC up to day 2. Starting on day 3, dilutions were needed on days 5 and 6 to keep the controls at maximum cell density. On day 7, Erlenmeyer controls were discontinued, as cell growth and maintenance were different from the CMBs, making T_{z} production comparisons unviable. As for the incubator CMB controls, one (CMB1) was discarded on day 5 due to emptying after a connection became loose. CMB2 and CMB3 were discarded on day 10 due to low viability. Due to firmware issues, the hoses for Ceibo5 and Ceibo5 disconnected during the autorun operation (after day 7 and 9), causing the CMBs to empty and ending the test for these systems. Ceibo4 was run for 12 days. After 12 days, due to a collapsed connection caused by excessive hose entanglement, the test was ceased. On day 4, autoruns were started on the Ceibo systems every 2 hours to refresh the medium and promote cell growth. On day 5, the autoruns were stopped, and CMB volumes were restored before sampling. On day 7, autoruns were set to every 55 and 40 minutes for Ceibo4 and Ceibo6, respectively, to maintain a maximum cell density of 8×10⁶ cells/mL. Ceibo5, with a lower VCC, had its autorun set to every 75 minutes on day 8. On day 9, Ceibo4 and Ceibo5 were set to autorun every 92 minutes. On day 10, Ceibo 4 was not sampled due to a clogged or severely constricted hose from entanglement. On day 11, Ceibo 4 reached a cell concentration of 9.59×10⁶ cells/mL with 96% viability. The autorun frequency was maintained to observe the maximum cell density achieved while maintaining good viability. On day 12, sampling was not conducted on Ceibo 4 due to a collapsed hose from entanglement.

FIG. 10C shows average cell viability percentage over time (days) of CHO cells for Ceibo systems and control tests: Erlenmeyer controls in an incubator (control, solid circles), incubator CMBs (solid squares), and Ceibos (Ceibo4 triangles, Ceibo5 upside down triangles, and Ceibo6 diamonds). Open circles indicate hose entanglement events that caused a setup termination; D marks days where control dilutions were performed. A(X′)^{Y} indicates the start of autorun in Ceibos, with X as the wait time in minutes between each autorun and Y as the Ceibo system identifier.

The desired maximum cell density was achieved in Ceibo4 and Ceibo6 on days 11 and 7, respectively. Ceibo5 was close to reaching DM but was terminated early. The incubator CMBs achieved lower VCC and cell viability throughout the test. The comparisons between the systems may be affected by differing maintenance, handling, and connections.

Despite the early test termination, cell growth in the Ceibo CMBs was observed, displaying similar growth to controls until day 2, then appearing to plateau until day 4, when autorun began in all systems. Cells showed an adaptation period, after which growth proceeded as expected, doubling each day and requiring less frequent autorun to maintain the established maximum cell density.

### Example 3: Cell Growth in a Cell Culture System

This example demonstrates the functioning of the bioreactor and cell culture system disclosed in the present disclosure. A maximum cell density of 8×10⁶ cells/mL was achieved and maintained for at least 15 days. Growth of Chinese Hamster Ovary (CHO) cells was achieved, and the cells produced trastuzumab.

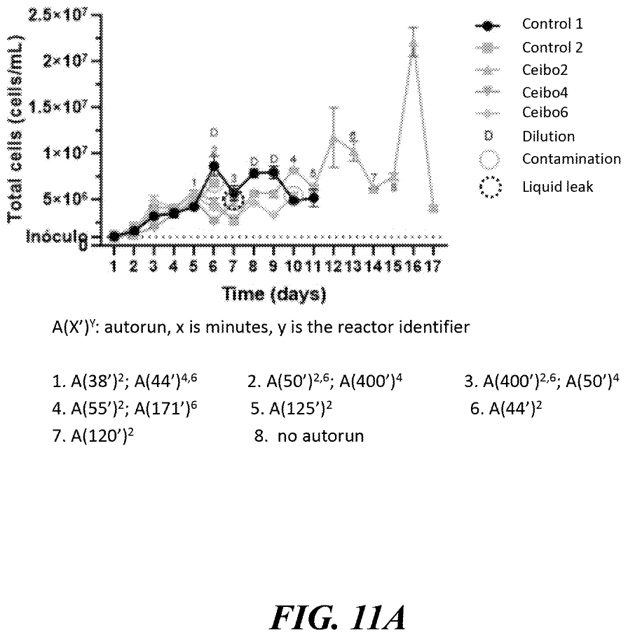

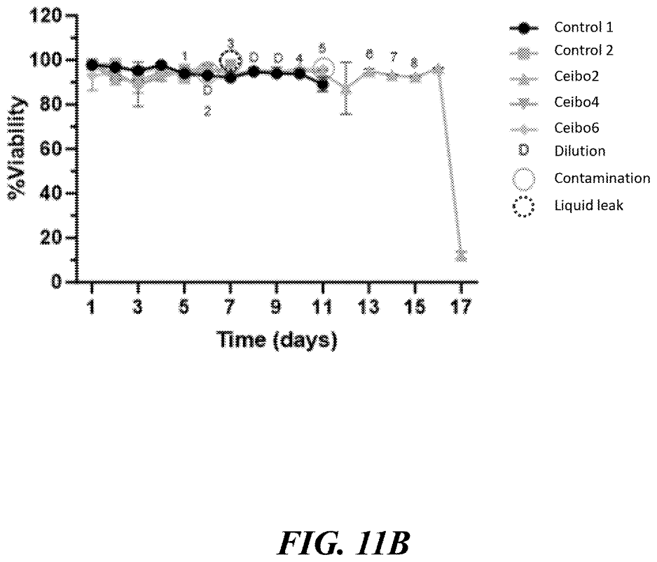

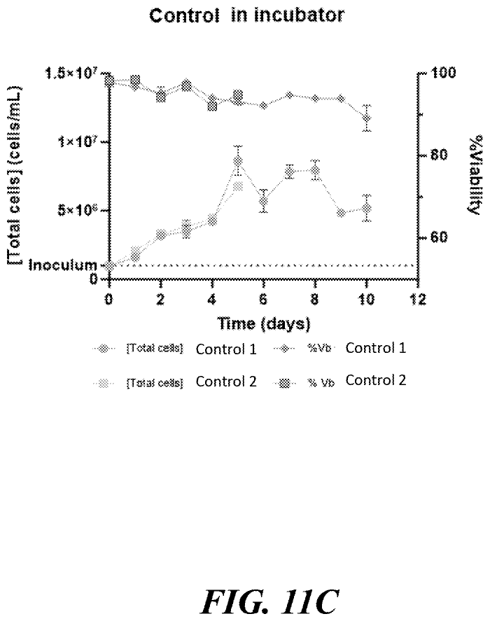

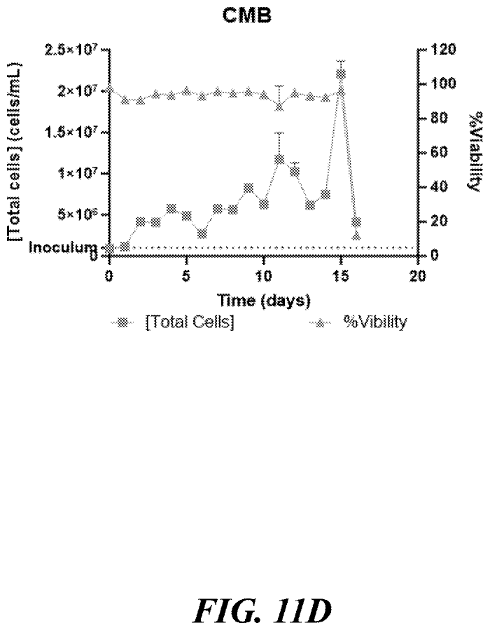

Three bioreactors (CMB2, CMB4, CMB6) were placed in three cell culture systems (Ceibo2, Ceibo4, Ceibo6) respectively. Control tests with two CMB bioreactors (control 1, control 2) were carried out in a commercial Numak incubator (37° C., 5% CO₂, 95% humidity). The production procedure was described in Example 2. Starting day 5, autoruns were started on the Ceibo systems to refresh the medium and promote cell growth. The autorun sequences were shown in FIG. 11A. A(X′)^{Y} indicates the start of autorun in Ceibos, with X as the wait time in minutes between each autorun and Y as the Ceibo system identifier.

FIG. 11A shows total cells over time (days) of CHO cells for Ceibo systems and control tests. FIG. 11B shows cell viability over time (days) of CHO cells for Ceibo systems and control tests. FIG. 11C shows total cells and cell viability for control 1 and control 2. FIG. 11D shows total cells and cell viability for Ceibo2. Control 2 had potential contamination at day 6 and the production was terminated. Ceibo6 had potential contamination at day 10 and the production was terminated. Ceibo4 had potential liquid leak at day 7 and the production was terminated. Dilution was performed for control 1 at day 6, 8, and 9. Control 1 was terminated at day 11 due to the reduction of the cell viability. Ceibo2 was run for 17 days. Ceibo2 achieved more than 90% cell viability for at least 16 days.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A bioreactor, comprising:
an inlet configured to receive a culture medium and a plurality of cells;
a first vertical wall and a second vertical wall defining a cell culture chamber; wherein said first vertical wall comprises a cavity configured to hold said culture medium and said plurality of cells; wherein said second vertical wall comprises a gas permeable membrane; and
an outlet configured to direct at least a portion of said culture medium and said plurality of cells out of said cell culture chamber,
wherein said cavity has a shape of U or intertwined U.

2. The bioreactor of claim 1, wherein said cavity has a volume of about 5 mL to about 60 mL.

3. The bioreactor of claim 1, wherein said cavity has a volume of about 6 mL to about 15 mL.

4. The bioreactor of any one of claims 1-3, wherein said membrane comprises a material selected from the group consisting of polydimethylsiloxane (PDMS), Polytetrafluoroethylene (PTFE), silicon, and coated fabric.

5. The bioreactor of any one of claims 1-4, wherein said membrane has a thickness of about 10 micrometers to 500 micrometers.

6. The bioreactor of any one of claims 1-5, wherein said cavity has a depth of about 1 mm to about 6 mm.

7. The bioreactor of any one of claims 1-6, wherein said second vertical wall comprises a support member, said support member having sufficient stiffness for providing support for said gas permeable membrane.

8. The bioreactor of claim 7, wherein said support member comprises a material selected from the group consisting of polycarbonate, polymethylmethacrylate and polyvinylchloride.

9. The bioreactor of claim 7, wherein said support member comprises a plurality of holes.

10. The bioreactor of any one of claims 1-9, further comprising a third vertical wall, wherein said second vertical wall and said third vertical wall define a gas chamber.

11. The bioreactor of claim 10, wherein said third vertical wall comprises a plurality of cavities.

12. The bioreactor of claim 10, further comprising a gas inlet and a gas outlet connected to said gas chamber.

13. The bioreactor of claim 1, further comprising a plurality of sensors.

14. The bioreactor of claim 13, wherein said plurality of sensors are configured to measure a temperature, medium volume, glucose, glutamate, lactate, pH, ions, osmolarity, cell productivity, cell viability, cell density, pressure, dissolved oxygen level, and/or dissolved CO₂ level inside said cell culture chamber.

15. A bioreactor, comprising:
an inlet configured to receive a culture medium and a plurality of cells;
a first vertical wall and a second vertical wall defining a cell culture chamber; wherein said first vertical wall comprises a cavity configured to hold said culture medium and said plurality of cells; wherein said second vertical wall comprises a gas permeable membrane; and
an outlet configured to direct at least a portion of said culture medium and said plurality of cells out of said cell culture chamber,
wherein said gas permeable membrane comprises a PTFE membrane.

16. The bioreactor of claim 15, wherein said cavity has a volume of about 5 mL to about 60 mL.

17. The bioreactor of claim 15, wherein said cavity has a volume of about 6 mL to about 15 mL.

18. The bioreactor of any one of claims 15-17, wherein said cavity has a shape that is U, intertwined U, disc-like, or oval.

19. The bioreactor of any one of claims 15-18, wherein said membrane has a thickness of about 10 micrometers to 500 micrometers.

20. The bioreactor of any one of claims 15-19, wherein said cavity has a depth of about 1 mm to about 6 mm.

21. The bioreactor of any one of claims 15-20, wherein said second vertical wall comprises a support member, said support member having sufficient stiffness for providing support for said gas permeable membrane.

22. The bioreactor of claim 21, wherein said support member comprises polycarbonate.

23. The bioreactor of claim 21, wherein said support member comprises a plurality of holes.

24. The bioreactor of any one of claims 15-23, further comprising a third vertical wall, wherein said second vertical wall and said third vertical wall define a gas chamber.

25. The bioreactor of claim 24, wherein said third vertical wall comprises a plurality of cavities.

26. The bioreactor of claim 24, further comprising a gas inlet and a gas outlet connected to said gas chamber.

27. The bioreactor of any one of claims 15-26, further comprising a plurality of sensors.

28. The bioreactor of claim 27, wherein said plurality of sensors are configured to measure a temperature, medium volume, glucose, glutamate, lactate, pH, ions, osmolarity, cell productivity, cell viability, cell density, pressure, dissolved oxygen level, and/or dissolved CO₂ level inside said cell culture chamber.

29. A bioreactor, comprising:
an inlet configured to receive a culture medium and a plurality of cells;
a first vertical wall and a second vertical wall defining a cell culture chamber; wherein said first vertical wall comprises a cavity configured to hold said culture medium and said plurality of cells; wherein said second vertical wall comprises a gas permeable membrane; and
an outlet configured to direct at least a portion of said culture medium and said plurality of cells out of said cell culture chamber,
wherein said cavity is configured for minimal dead zone.

30. The bioreactor of claim 29, wherein said cavity has a volume of about 5 mL to about 60 mL.

31. The bioreactor of claim 29, wherein said cavity has a volume of about 6 mL to about 15 mL.

32. The bioreactor of any one of claims 29-31, wherein said cavity has a shape of U, intertwined U, disc-like, or oval.

33. The bioreactor of any one of claims 29-32, wherein said membrane comprises a material selected from the group consisting of polydimethylsiloxane (PDMS), Teflon, Polytetrafluoroethylene (PTFE), silicone, and coated fabric.

34. The bioreactor of any one of claims 29-33, wherein said membrane has a thickness of about 10 micrometers to 500 micrometers.

35. The bioreactor of any one of claims 29-34, wherein said cavity has a depth of about 1 mm to about 6 mm.

36. The bioreactor of any one of claims 29-35, wherein said second vertical wall comprises a support member, said support member having sufficient stiffness for providing support for said gas permeable membrane.

37. The bioreactor of claim 36, wherein said support member comprises polycarbonate.

38. The bioreactor of claim 36, wherein said support member comprises a plurality of holes.

39. The bioreactor of any one of claims 29-38, further comprising a third vertical wall, wherein said second vertical wall and said third vertical wall define a gas chamber.

40. The bioreactor of claim 39, wherein said third vertical wall comprises a plurality of cavities.

41. The bioreactor of claim 39, further comprising a gas inlet and a gas outlet connected to said gas chamber.

42. The bioreactor of any one of claims 29-41, further comprising a plurality of sensors.

43. The bioreactor of claim 42, wherein said plurality of sensors are configured to measure a temperature, medium volume, glucose, glutamate, lactate, pH, ions, osmolarity, cell productivity, cell viability, cell density, pressure, dissolved oxygen level, and/or dissolved CO₂ level inside said cell culture chamber.

44. A cell culture system, comprising:
at least one bioreactor of any one of claims 1-43;
at least one flipper module configured to rotate said at least one bioreactor; and
a harvest and feed module configured to supply a culture medium to said at least one bioreactor and/or take a sample from said at least one bioreactor for analysis.

45. The cell culture system of claim 44, further comprises an additional sensor configured to measure a temperature and/or humidity of said cell culture system.

46. The cell culture system of claim 44 or 45, wherein one flipper module of said at least one flipper module is configured to rotate one bioreactor of said at least one bioreactor along a horizontal axis.

47. The cell culture system of any one of claims 44-46, further comprising a temperature controller.

48. The cell culture system of any one of claims 44-47, further comprising a heater.

49. The cell culture system of any one of claims 44-48, wherein said harvest and feed module comprises a tubing support, a L shaped enclosure, a plurality of microchannels, and a plurality of valve caps.

50. The cell culture system of any one of claims 44-49, wherein said harvest and feed module comprises a plurality of IR sensors configured to sense a change in the interface.

51. The cell culture system of claim 50, wherein said plurality of sensors are further configured to monitor a sampling volume.

52. The cell culture system of claim 50, wherein said harvest and feed module is connected to said at least one bioreactor.

53. A method of growing cells, comprising:
(a) inoculating a bioreactor of any one of claims 1-43 with a cell medium comprising at least one cell, and
(b) incubating said bioreactor for a period of time under conditions sufficient to permitting cell growth;
wherein during said incubating, rotating said bioreactor for a pre-determined angle at pre-determined time periods.

54. The method of claim 53, wherein said rotating is along a horizontal axis.

55. The method of claim 53 or 54, wherein said rotating comprises (i) rotating said bioreactor by 180° at 30°/s and holding said bioreactor for 4 seconds, (ii) rotating back to original position at 30°/s in a same or opposite direction and holding said bioreactor for 4 s, (iii) rotating said bioreactor at 30°/s by 180° in in a same or opposite direction and holding said bioreactor for 4 s, (iv) rotating back at 30°/s to original position in a same or opposite direction and holding said bioreactor for 4 s, and (v) repeating (i)-(iv).

56. The method of claim 53 or 54, wherein said rotating comprises (i) rotating said bioreactor by 180° at 60°/s and holding said bioreactor for 4 seconds, (ii) rotating back to original position at 60°/s in a same or opposite direction and holding said bioreactor for 4 s, (iii) rotating said bioreactor at 60°/s by 180° in a same or opposite direction and holding said bioreactor for 4 s, (iv) rotating back at 60°/s to original position in a same or opposite direction and holding said bioreactor for 4 s, and (v) repeating (i)-(iv).

57. The method of claim 53 or 54, wherein said rotating comprises (i) rotating said bioreactor by 180° at 15°/s and holding said bioreactor for 4 seconds, (ii) rotating back to original position at 15°/s in a same or opposite direction and holding said bioreactor for 4 s, (iii) rotating said bioreactor at 15°/s by 180° in a same or opposite direction and holding said bioreactor for 4 s, (iv) rotating back at 15°/s to original position in a same or opposite direction and holding said bioreactor for 4 s, and (v) repeating (i)-(iv).

58. The method of any one of claims 53-57, wherein said cell medium is at most 80% volume of said cell culture chamber.

59. The method of any one of claims 53-58, wherein said period of time is from 1 day to 30 days.

60. The method of any one of claims 53-59, further comprising taking a sample from said bioreactor and determining a cell density.

61. The method of claim 60, further comprising ceasing said incubating when said cell density reaches a pre-determined value.
